(19) United States
(12) Patent Application Publication
(10) Pub. No.: US 2021/0059569 A1
(43) Pub. Date: Mar. 4, 2021

(54) FALL RISK EVALUATION METHOD, FALL RISK EVALUATION DEVICE, AND NON-TRANSITORY COMPUTER-READABLE RECORDING MEDIUM IN WHICH FALL RISK EVALUATION PROGRAM IS RECORDED

(71) Applicant: Panasonic Intellectual Property Corporation of America, Torrance, CA (US)

(72) Inventors: Takahiro HIYAMA, Tokyo (JP); Yoshikuni SATO, Tokyo (JP); Takahiro AIHARA, Osaka (JP); Kengo WADA, Osaka (JP); Taichi HAMATSUKA, Osaka (JP); Yoshihiro MATSUMURA, Osaka (JP)

(21) Appl. No.: 16/998,482

(22) Filed: Aug. 20, 2020

Related U.S. Application Data
(60) Provisional application No. 62/893,304, filed on Aug. 29, 2019.

(30) Foreign Application Priority Data
Feb. 14, 2020 (JP) 2020-023433

(51) Int. Cl. A61B 5/11; A61B 5/00

(52) U.S. Cl. CPC A61B 5/1117; A61B 5/112; A61B 5/742; A61B 5/7275; A61B 5/1127

(57) ABSTRACT

A fall risk evaluation method in a fall risk evaluation device that evaluates a fall risk based on a walking motion of a subject includes: acquiring walking data related to walking of the subject; detecting, from the walking data, at least one walking parameter of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase; and determining a fall risk of the subject by using at least one walking parameter.

### FIELD OF THE INVENTION

The present disclosure relates to a technology for evaluating the fall risk based on walking motion of a subject.

### BACKGROUND ART

In recent years, in order to grasp the health condition of the elderly, a technology for easily estimating a physical function has been developed. In particular, the elderly are highly likely to fall due to decline in physical function, which may cause fracture or bedridden state. Therefore, it is necessary to find elderly people who are prone to fall, i.e., elderly people who have a fall risk, as early as possible, and to take measures to prevent them from falling.

Conventionally, technologies have been proposed for evaluating cognitive functions or motor functions based on parameters measured from daily walking.

For example, Japanese Patent Application Laid-Open No. 2013-255786 discloses a method for evaluating the likelihood of a senile disorder (senile disorder risk) based on walking parameters measured by walking.

In Japanese Patent Application Laid-Open No. 2018-114319, for example, acceleration sensor attached to the waist of a subject measures a longitudinal acceleration, a lateral acceleration, and a vertical acceleration during the movement of the subject, and the movement ability is evaluated based on temporal changes in the longitudinal acceleration, the lateral acceleration, and the vertical acceleration.

However, with the above-mentioned conventional technologies, it is difficult to easily and highly accurately evaluate the fall risk, and further improvement has been required.

### SUMMARY OF THE INVENTION

The present disclosure has been made to solve the above problems, and an object of the present disclosure is to provide a technology capable of easily and highly accurately evaluating the fall risk.

A fall risk evaluation method according to an aspect of the present disclosure is a fall risk evaluation method in a fall risk evaluation device that evaluates a fall risk based on the walking motion of a subject, the fall risk evaluation method including: acquiring walking data related to walking of the subject; detecting, from the walking data, at least one of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase; and determining a fall risk of the subject by using at least one of the vertical displacement of the waist in the stance phase, the vertical displacement of the waist in the swing phase, the angle of the knee joint in the stance phase, and the angle of the ankle joint in the swing phase.

### BRIEF DESCRIPTION OF THE DRAWINGS

FIG. 1 is a block diagram showing a configuration of a fall risk evaluation system in an embodiment of the present disclosure;

FIG. 2 is a view for explaining processing of extracting skeleton data from two-dimensional image data in the present embodiment:

FIG. 3 is a view for explaining a walking cycle in the present embodiment;

FIG. 4 is a flowchart for explaining the fall risk evaluation processing using a walking motion of a subject in the present embodiment;

FIG. 5 is a flowchart for explaining the fall risk determination processing in step S4 of FIG. 4;

FIG. 6 is a flowchart for explaining another example of the fall risk determination processing in step S4 of FIG. 4:

FIG. 7 is a view showing a change in the vertical displacement of the waist in one walking cycle in the present embodiment:

FIG. 8 is a view showing an ROC curve obtained as a result of determining the presence or absence of a fall risk using a prediction model in the present embodiment:

FIG. 9 is a view showing an ROC curve obtained as a result of determining the presence or absence of a fall risk using a prediction model in a first modification of the present embodiment;

FIG. 10 is a view showing an average of mean values of time series data of the vertical displacement of the waist of subjects who do not have a fall risk in the period of 9% to 19% of one walking cycle and an average of mean values of time series data of the vertical displacement of the waist of subjects who have a fall risk in the period of 9% to 19% of one walking cycle in the first modification of the present embodiment:

FIG. 11 is a view showing an ROC curve obtained as a result of determining the presence or absence of a fall risk using a prediction model in a second modification of the present embodiment;

FIG. 12 is a view showing a change in the angle of one knee joint in one walking cycle in a third modification of the present embodiment:

FIG. 13 is a view showing an ROC curve obtained as a result of determining the presence or absence of a fall risk using a prediction model in the third modification of the present embodiment;

FIG. 14 is a view showing an ROC curve obtained as a result of determining the presence or absence of a fall risk using a prediction model in a fourth modification of the present embodiment;

FIG. 15 is a view showing an average of the angles of the knee joint of one leg of the subjects who do not have a fall risk at the time point of 35% of one walking cycle and an average of the angles of the knee joint of one leg of the subjects who have a fall risk at the time point of 35% of one walking cycle in the fourth modification of the present embodiment;

FIG. 16 is a view showing a change in the angle of one ankle joint in one walking cycle in a fifth modification of the present embodiment;

FIG. 17 is a view showing an ROC curve obtained as a result of determining the presence or absence of a fall risk using a prediction model in the fifth modification of the present embodiment;

FIG. 18 is a view showing an ROC curve obtained as a result of determining the presence or absence of a fall risk using a prediction model in a sixth modification of the present embodiment:

FIG. 19 is a view showing an average of mean values of time series data of the angle of the ankle joint of one foot of the subjects who do not have a fall risk in the period of 84% to 89% of one walking cycle and an average of mean values of time series data of the angle of the ankle joint of one foot of the subjects who have a fall risk in the period of 84% to 89% of one walking cycle in the sixth modification of the present embodiment;

FIG. 20 is a view showing an ROC curve obtained as a result of determining the presence or absence of a fall risk using a prediction model in a seventh modification of the present embodiment;

FIG. 21 is a view showing an ROC curve obtained as a result of determining the presence or absence of a fall risk using a prediction model in an eighth modification of the present embodiment;

FIG. 22 is a view showing an ROC curve obtained as a result of determining the presence or absence of a fall risk using a prediction model in a ninth modification of the present embodiment;

FIG. 23 is a view showing an ROC curve obtained as a result of determining the presence or absence of a fall risk using a prediction model in a tenth modification of the present embodiment;

FIG. 24 is a view showing an ROC curve obtained as a result of determining the presence or absence of a fall risk using a prediction model in an eleventh modification of the present embodiment; and

FIG. 25 is a view showing an example of an evaluation result screen displayed in the present embodiment.

### DESCRIPTION OF EMBODIMENTS

(Findings on which the Present Disclosure is Based)

A sheet type pressure sensor or a three-dimensional motion analysis system is used for measurement of a walking parameter in Japanese Patent Application Laid-Open No. 2013-255786. The sheet type pressure sensor measures a pressure distribution at the time of walking, and measures a walking parameter from the pressure distribution. A three-dimensional motion analysis system measures a walking parameter by acquiring, from a plurality of video cameras, image information in which a marker attached on a foot is captured, and analyzing the motion from the image information. It requires a great amount of time and effort to install such a sheet type pressure sensor or a three-dimensional motion analysis system. Therefore, with Japanese Patent Application Laid-Open No. 2013-255786, it is difficult to easily evaluate the senile disorder risk.

Furthermore, walking parameters used in Japanese Patent Application Laid-Open No. 2013-255786 are two or more selected from a cadence, a stride, a walking ratio, a step, a walking interval, a walking angle, a toe angle, a stride right-and-left difference, a walking interval right-and-left difference, a walking angle right-and-left difference, and both legs support period right-and-left difference. The walking angle is an angle formed by a straight line connecting one of the right and left heels with the other heel and the travel direction. The toe angle is an angle formed by a straight line connecting the heel with the toe and the travel direction. Furthermore, in Japanese Patent Application Laid-Open No. 2013-255786, the senile disorder risk of a senile disorder selected from at least knee pain, lower back pain, incontinence of urine, dementia, and sarcopenia is evaluated. However, Japanese Patent Application Laid-Open No. 2013-255786 does not disclose evaluating a senile disorder risk using another walking parameter, and there is a possibility that the use of another walking parameter further improves the evaluation accuracy of the senile disorder risk.

The moving ability evaluation device in Japanese Patent Application Laid-Open No. 2018-114319 evaluates at least one of longitudinal balance, body weight movement, and lateral balance when a subject is moving, from longitudinal acceleration, lateral acceleration, and vertical acceleration when the subject is moving. However, Japanese Patent Application Laid-Open No. 2018-114319 does not disclose evaluating the fall risk using another parameter, and there is a possibility that the use of another walking parameter further improves the evaluation accuracy of the fall risk.

In order to solve the above problems, the fall risk evaluation method according to an aspect of the present disclosure is a fall risk evaluation method in a fall risk evaluation device that evaluates a fall risk based on the walking motion of a subject, the fall risk evaluation method including: acquiring walking data related to walking of the subject; detecting, from the walking data, at least one of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase; and determining a fall risk of the subject by using at least one of the vertical displacement of the waist in the stance phase, the vertical displacement of the waist in the swing phase, the angle of the knee joint in the stance phase, and the angle of the ankle joint in the swing phase.

According to this configuration, at least one of the vertical displacement of the waist in the stance phase of one leg of a walking subject, the vertical displacement of the waist in the swing phase of the one leg, the angle of the knee joint of one leg in the stance phase, and the angle of the ankle joint of one foot in the swing phase is used as a parameter correlated with the fall risk of the subject. Walking motion of subjects who have a fall risk tends to be different from walking motion of subjects who do not have a fall risk. In this manner, since fall risk of the subject is determined using a parameter correlated with the fall risk of a walking subject, the fall risk of the subject can be evaluated with high accuracy.

Furthermore, a large-scale device is unnecessary because at least one of the vertical displacement of the waist in the stance phase of one leg of a walking subject the vertical displacement of the waist in the swing phase of the one leg, the angle of the knee joint of one leg in the stance phase, and the angle of the ankle joint of one foot in the swing phase can be easily detected from image data obtained by capturing an image of a walking subject, for example. Therefore, this configuration can easily evaluate the fall risk of a subject.

In addition, in the fall risk evaluation method described above, in the detection, time series data of the vertical displacement of the waist in a predetermined period of the stance phase may be detected, and in the determination, the fall risk of the subject may be determined by using a mean value of the time series data of the vertical displacement of the waist.

There is a significant difference in vertical displacement of the waist in a predetermined period of the stance phase of one leg of a walking subject between subjects who have a fall risk and subjects who do not have a fall risk. Therefore, according to this configuration, the fall risk of the subject can be reliably evaluated by using a mean value of time series data of the vertical displacement of the waist in a predetermined period of the stance phase of one leg of a walking subject.

Furthermore, in the above-described fall risk evaluation method, on the condition that a period from when one foot of the subject touches the ground to when the one foot touches the ground again is expressed as one walking cycle and the one walking cycle is expressed by 1% to 100%, the predetermined period may be a period of 1% to 60% of the one walking cycle.

According to the present configuration, the period from when one foot of the subject touches the ground to when the one foot touches the ground again is expressed as one walking cycle, and one walking cycle is expressed as 1% to 100%. At this time, the fall risk of the subject can be reliably evaluated by using a mean value of time series data of the vertical displacement of the waist in the period of 1% to 60% of one walking cycle.

Furthermore, in the above-described fall risk evaluation method, on the condition that a period from when one foot of the subject touches the ground to when the one foot touches the ground again is expressed as one walking cycle and the one walking cycle is expressed by 1% to 100%, the predetermined period may be a period of 9% to 19% of the one walking cycle.

According to this configuration, the fall risk of the subject can be more reliably evaluated by using a mean value of the time series data of the vertical displacement of the waist in the period of 9% to 19% of one walking cycle.

In addition, in the fall risk evaluation method described above, in the detection, time series data of the vertical displacement of the waist in a predetermined period of the swing phase may be detected, and in the determination, the fall risk of the subject may be determined by using a mean value of the time series data of the vertical displacement of the waist.

There is a significant difference in vertical displacement of the waist in a predetermined period of the swing phase of one leg of a walking subject between subjects who have a fall risk and subjects who do not have a fall risk. Therefore, according to this configuration, the fall risk of the subject can be reliably evaluated by using a mean value of time series data of the vertical displacement of the waist in a predetermined period of the swing phase of the one leg of a walking subject.

Furthermore, in the above-described fall risk evaluation method, on the condition that a period from when one foot of the subject touches the ground to when the one foot touches the ground again is expressed as one walking cycle and the one walking cycle is expressed by 1% to 100%, the predetermined period may be a period of 61% to 100% of the one walking cycle.

According to the present configuration, the period from when one foot of the subject touches the ground to when the one foot touches the ground again is expressed as one walking cycle, and one walking cycle is expressed as 1% to 100%. At this time, the fall risk of the subject can be reliably evaluated by using a mean value of time series data of the vertical displacement of the waist in the period of 61% to 100% of one walking cycle.

In addition, in the fall risk evaluation method described above, in the detection, time series data of an angle of the knee joint in a predetermined period of the stance phase may be detected, and in the determination, the fall risk of the subject may be determined by using a mean value of the time series data of the angle of the knee joint.

There is a significant difference in angle of the knee joint in a predetermined period of the stance phase of one leg of a walking subject between subjects who have a fall risk and subjects who do not have a fall risk. Therefore, according to this configuration, the fall risk of the subject can be reliably evaluated by using a mean value of time series data of the angle of the knee joint in a predetermined period of the stance phase of one leg of a walking subject.

Furthermore, in the above-described fall risk evaluation method, on the condition that a period from when one foot of the subject touches the ground to when the one foot touches the ground again is expressed as one walking cycle and the one walking cycle is expressed by 1% to 100%, the predetermined period may be a period of 1% to 60% of the one walking cycle.

According to the present configuration, the period from when one foot of the subject touches the ground to when the one foot touches the ground again is expressed as one walking cycle, and one walking cycle is expressed as 1% to 100%. At this time, the fall risk of the subject can be reliably evaluated by using a mean value of time series data of the angle of the knee joint in the period of 1% to 60% of one walking cycle.

Furthermore, in the above-described fall risk evaluation method, on the condition that a period from when one foot of the subject touches the ground to when the one foot touches the ground again is expressed as one walking cycle and the one walking cycle is expressed by 1% to 100%, in the determination, the fall risk of the subject may be determined by using the angle of the knee joint at the time point of 35% of the one walking cycle.

According to this configuration, the fall risk of the subject can be more reliably evaluated by using the angle of the knee joint at the time point of 35% of one walking cycle.

In addition, in the fall risk evaluation method described above, in the detection, time series data of the angle of the ankle joint in a predetermined period of the swing phase may be detected, and in the determination, the fall risk of the subject may be determined by using a mean value of the time series data of the angle of the ankle joint.

There is a significant difference in angle of the ankle joint in a predetermined period of the swing phase of one leg of a walking subject between subjects who have a fall risk and subjects who do not have a fall risk. Therefore, according to this configuration, the fall risk of the subject can be reliably evaluated by using a mean value of time series data of the angle of the ankle joint in a predetermined period of the swing phase of the one leg of a walking subject.

Furthermore, in the above-described fall risk evaluation method, on the condition that a period from when one foot of the subject touches the ground to when the one foot touches the ground again is expressed as one walking cycle and the one walking cycle is expressed by 1% to 100%, the predetermined period may be a period of 61% to 100% of the one walking cycle.

According to the present configuration, the period from when one foot of the subject touches the ground to when the one foot touches the ground again is expressed as one walking cycle, and one walking cycle is expressed as 1% to 100%. At this time, the fall risk of the subject can be reliably evaluated by using a mean value of time series data of the angle of the ankle joint in the period of 61% to 100% of one walking cycle.

Furthermore, in the above-described fall risk evaluation method, on the condition that a period from when one foot of the subject touches the ground to when the one foot touches the ground again is expressed as one walking cycle and the one walking cycle is expressed by 1% to 100%, the predetermined period may be a period of 84% to 89% of the one walking cycle.

According to this configuration, the fall risk of the subject can be more reliably evaluated by using a mean value of the time series data of the angle of the ankle joint in the period of 84% to 89% of one walking cycle.

In addition, in the fall risk evaluation method described above, in the detection, time series data of the vertical displacement of the waist in a first period of the stance phase and time series data of the angle of the knee joint in a second period of the stance phase may be detected, and in the determination, the fall risk of the subject may be determined by using a mean value of the time series data of the vertical displacement of the waist in the first period and a mean value of the time series data of the angle of the knee joint in the second period.

According to this configuration, a mean value of the time series data of the vertical displacement of the waist in the first period of the stance phase of one leg and a mean value of the time series data of the angle of the knee joint in the second period of the stance phase of one leg are used in combination, whereby the fall risk can be evaluated more accurately than by using each of them in isolation.

In addition, in the fall risk evaluation method described above, in the detection, time series data of the vertical displacement of the waist in a first period of the stance phase, time series data of the vertical displacement of the waist in a second period of the swing phase, and time series data of the angle of the ankle joint in a third period of the swing phase may be detected, and in the determination, the fall risk of the subject may be determined by using a mean value of the time series data of the vertical displacement of the waist in the first period, a mean value of the time series data of the vertical displacement of the waist in the second period, and a mean value of the time series data of the angle of the ankle joint in the third period.

According to this configuration, a mean value of the time series data of the vertical displacement of the waist in the first period of the stance phase of one leg, a mean value of the time series data of the vertical displacement of the waist in the second period of the swing phase of one leg, and a mean value of the time series data of the angle of the ankle joint in the third period of the swing phase of one leg are used in combination, whereby the fall risk can be evaluated more accurately than by using each of them in isolation.

In addition, in the fall risk evaluation method described above, in the detection, time series data of the angle of the knee joint in a first period of the stance phase and time series data of the angle of the ankle joint in a second period of the swing phase may be detected, and in the determination, the fall risk of the subject may be determined by using a mean value of the time series data of the angle of the knee joint in the first period and a mean value of the time series data of the angle of the ankle joint in the second period.

According to this configuration, a mean value of the time series data of the angle of the knee joint in the first period of the stance phase of one leg and a mean value of the time series data of the angle of the ankle joint in the second period of the swing phase of one leg are used in combination, whereby the fall risk can be evaluated more accurately than by using each of them in isolation.

In addition, in the fall risk evaluation method described above, in the detection, time series data of the vertical displacement of the waist in a first period of the stance phase, time series data of the angle of the knee joint in a second period of the stance phase, and time series data of the angle of the ankle joint in a third period of the swing phase may be detected, and in the determination, the fall risk of the subject may be determined by using a mean value of the time series data of the vertical displacement of the waist in the first period, a mean value of time series data of the angle of the knee joint in the second period, and a mean value of time series data of the angle of the ankle joint in the third period.

According to this configuration, a mean value of the time series data of the vertical displacement of the waist in the first period of the stance phase of one leg, a mean value of the time series data of the angle of the knee joint in the second period of the stance phase of one leg, and a mean value of the time series data of the angle of the ankle joint in the third period of the swing phase of one leg are used in combination, whereby the fall risk can be evaluated more accurately than by using each of them in isolation.

In addition, in the fall risk evaluation method described above, in the determination, it may be determined that the subject has the fall risk when the vertical displacement of the waist in the stance phase is smaller than a threshold value, when the vertical displacement of the waist in the swing phase is smaller than a threshold value, when the angle of the knee joint in the stance phase is smaller than a threshold value, or when the angle of the ankle joint in the swing phase is smaller than a threshold value.

According to this configuration, it is determined that the subject has the fall risk when the vertical displacement of the waist in the stance phase is smaller than the threshold value, when the vertical displacement of the waist in the swing phase is smaller than the threshold value, when the angle of the knee joint in the stance phase is smaller than the threshold value, or when the angle of the ankle joint in the swing phase is smaller than the threshold value. Accordingly, by comparing the vertical displacement of the waist in the stance phase, the vertical displacement of the waist in the swing phase, the angle of the knee joint in the stance phase, or the angle of the ankle joint in the swing phase with the threshold value, it is possible to easily determine whether or not the subject has a fall risk.

In addition, in the fall risk evaluation method described above, in the determination, whether or not the subject has the fall risk may be determined by inputting at least one of the vertical displacement of the waist in the stance phase, the vertical displacement of the waist in the swing phase, the angle of the knee joint in the stance phase, and the angle of the ankle joint in the swing phase that have been detected into a prediction model generated with at least one of the vertical displacement of the waist in the stance phase, the vertical displacement of the waist in the swing phase, the angle of the knee joint in the stance phase, and the angle of the ankle joint in the swing phase as an input value and with whether or not the subject has the fall risk as an output value.

According to this configuration, the prediction model is generated with at least one of the vertical displacement of the waist in the stance phase, the vertical displacement of the waist in the swing phase, the angle of the knee joint in the stance phase, and the angle of the ankle joint in the swing phase as an input value, and with whether or not the subject has a fall risk as an output value. Then, whether or not the subject has a fall risk is determined by inputting, into the prediction model, at least one of the vertical displacement of the waist in the stance phase, the vertical displacement of the waist in the swing phase, the angle of the knee joint in the stance phase, and the angle of the ankle joint in the swing phase that have been detected. Accordingly, by storing the prediction model in advance, it is possible to easily determine whether or not the subject has a fall risk.

A fall risk evaluation device according to another aspect of the present disclosure is a fall risk evaluation device that evaluates the fall risk based on the walking motion of a subject, the fall risk evaluation device including: an acquisition unit that acquires walking data related to walking of the subject; a detection unit that detects, from the walking data, at least one of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase; and a determination unit that determines a fall risk of the subject by using at least one of the vertical displacement of the waist in the stance phase, the vertical displacement of the waist in the swing phase, the angle of the knee joint in the stance phase, and the angle of the ankle joint in the swing phase.

According to this configuration, at least one of the vertical displacement of the waist in the stance phase of one leg of a walking subject, the vertical displacement of the waist in the swing phase of the one leg, the angle of the knee joint of one leg in the stance phase, and the angle of the ankle joint of one foot in the swing phase is used as a parameter correlated with the fall risk of the subject. Walking motion of subjects who have a fall risk tends to be different from walking motion of subjects who do not have a fall risk. In this manner, since fall risk of the subject is determined using a parameter correlated with the fall risk of a walking subject, the fall risk of the subject can be evaluated with high accuracy.

Furthermore, a large-scale device is unnecessary because at least one of the vertical displacement of the waist in the stance phase of one leg of a walking subject, the vertical displacement of the waist in the swing phase of the one leg, the angle of the knee joint of one leg in the stance phase, and the angle of the ankle joint of one foot in the swing phase can be easily detected from image data obtained by capturing an image of a walking subject, for example. Therefore, this configuration can easily evaluate the fall risk of a subject.

A non-transitory computer-readable recording medium in which a fall risk evaluation program is recorded according to another aspect of the present disclosure is a non-transitory computer-readable recording medium in which the fall risk evaluation program that evaluates the fall risk based on walking motion of a subject is recorded, in which a computer is caused to function so as to acquire walking data related to walking of the subject, detect, from the walking data, at least one of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase, and determine a fall risk of the subject by using at least one of the vertical displacement of the waist in the stance phase, the vertical displacement of the waist in the swing phase, the angle of the knee joint in the stance phase, and the angle of the ankle joint in the swing phase.

According to this configuration, at least one of the vertical displacement of the waist in the stance phase of one leg of a walking subject, the vertical displacement of the waist in the swing phase of the one leg, the angle of the knee joint of one leg in the stance phase, and the angle of the ankle joint of one foot in the swing phase is used as a parameter correlated with the fall risk of the subject. Walking motion of subjects who have a fall risk tends to be different from walking motion of subjects who do not have a fall risk. In this manner, since fall risk of the subject is determined using a parameter correlated with the fall risk of a walking subject, the fall risk of the subject can be evaluated with high accuracy.

Furthermore, a large-scale device is unnecessary because at least one of the vertical displacement of the waist in the stance phase of one leg of a walking subject, the vertical displacement of the waist in the swing phase of the one leg, the angle of the knee joint of one leg in the stance phase, and the angle of the ankle joint of one foot in the swing phase can be easily detected from image data obtained by capturing an image of a walking subject, for example. Therefore, this configuration can easily evaluate the fall risk of a subject.

An embodiment of the present disclosure will now be described with reference to the accompanying drawings. It is to be noted that the following embodiment is an example embodying the present disclosure, and does not limit the technical scope of the present disclosure.

### Embodiment

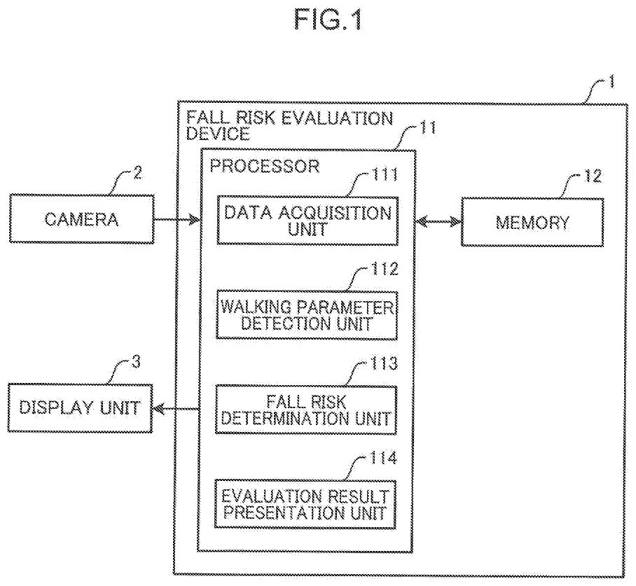

A fall risk evaluation system according to the present embodiment will be described below with reference to FIG. 1.

FIG. 1 is a block diagram showing a configuration of a fall risk evaluation system in an embodiment of the present disclosure.

The fall risk evaluation system shown in FIG. 1 includes a fall risk evaluation device 1, a camera 2, and a display unit 3.

The camera 2 captures an image of a walking subject. The camera 2 outputs moving image data showing a walking subject to the fall risk evaluation device 1. The camera 2 is connected with the fall risk evaluation device 1 by wire or wirelessly.

The fall risk evaluation device 1 includes a processor 11 and a memory 12.

The processor 11 is, for example, a central processing unit (CPU), and includes a data acquisition unit 111, a walking parameter detection unit 112, a fall risk determination unit 113, and an evaluation result presentation unit 114.

The memory 12 is a storage device capable of storing various kinds of information, such as a random access memory (RAM), a hard disk drive (HDD), a solid state drive (SSD), or a flash memory.

The data acquisition unit 111 acquires walking data related to walking of the subject. The walking data is moving image data obtained by capturing an image of a walking subject, for example. The data acquisition unit 111 acquires moving image data having been output by the camera 2.

The walking parameter detection unit 112 extracts skeleton data showing the skeleton of the subject from moving image data acquired by the data acquisition unit 11. The skeleton data is represented by coordinates of a plurality of feature points indicating the joints and the like of the subject and straight lines connecting the feature points. The walking parameter detection unit 112 may use software (e.g., OpenPose or 3D-pose-baseline) that detects the coordinates of feature points of a person from two-dimensional image data.

The processing of extracting skeleton data from two-dimensional image data will now be described.

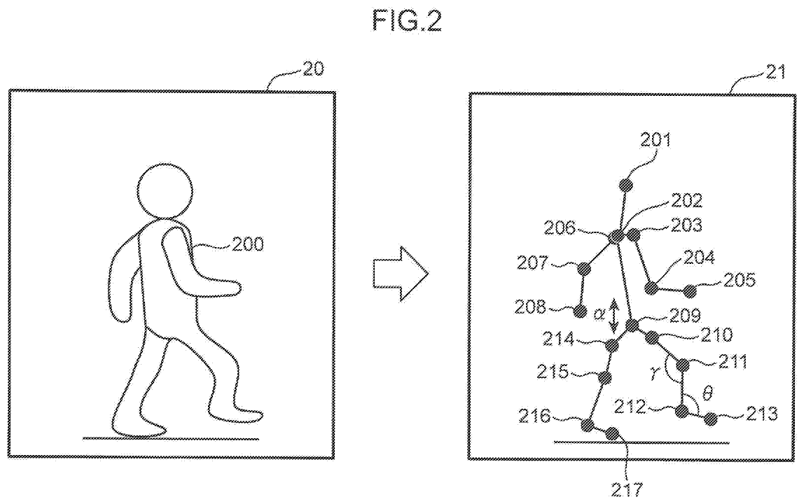

FIG. 2 is a view for explaining processing of extracting skeleton data from two-dimensional image data in the present embodiment.

The walking parameter detection unit 112 extracts skeleton data 21 from two-dimensional image data 20 including an image of a walking subject 200. The skeleton data 21 includes a feature point 201 indicating the head, a feature point 202 indicating the center of both shoulders, a feature point 203 indicating the right shoulder, a feature point 204 indicating the right elbow, a feature point 205 indicating the right hand, a feature point 206 indicating the left shoulder, a feature point 207 indicating the left elbow, a feature point 208 indicating the left hand, a feature point 209 indicating the waist, a feature point 210 indicating the right hip joint, a feature point 211 indicating the right knee joint, a feature point 212 indicating the right ankle joint, a feature point 213 indicating the right toe, a feature point 214 indicating the left hip joint, a feature point 215 indicating the left knee joint, a feature point 216 indicating the left ankle joint, and a feature point 217 indicating the left toe.

The moving image data is composed of a plurality of two-dimensional image data. The walking parameter detection unit 112 extracts time series skeleton data from each of a plurality of two-dimensional image data constituting moving image data. It is to be noted that the walking parameter detection unit 112 may extract skeleton data from two-dimensional image data of all frames or may extract skeleton data from two-dimensional image data of each predetermined frame. In addition, in the present embodiment, the fall risk is evaluated based on the movement of mainly the lower limbs of the walking subject. Therefore, the walking parameter detection unit 112 may extract only the skeleton data of the lower limbs of the subject.

In addition, the walking parameter detection unit 112 clips skeleton data corresponding to one walking cycle of the subject from time series skeleton data extracted from moving image data. The human walking motion is a cyclic motion.

The walking cycle of the subject will now be described.

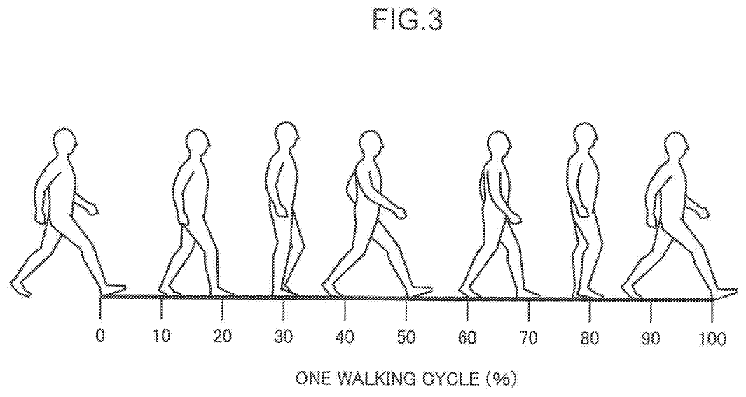

FIG. 3 is a view for explaining a walking cycle in the present embodiment.

As shown in FIG. 3, the period from when one foot of the subject touches the ground to when the one foot touches the ground again is expressed as one walking cycle. The one walking cycle shown in FIG. 3 is a period from when the right foot of the subject touches the ground to when the right foot touches the ground again. In addition, one walking cycle is normalized to 1% to 100%. The period of 1% to 60% of one walking cycle is called a stance phase in which one foot (e.g., right foot) is on the ground, and the period of 61% to 100% of one walking cycle is called a swing phase in which one foot (e.g., right foot) is off the ground. One walking cycle includes the stance phase and the swing phase. It is to be noted that one walking cycle may be a period from when the left foot of the subject touches the ground to when the left foot touches the ground again.

The walking parameter detection unit 112 detects, from walking data, at least one of a vertical displacement of the waist of the subject in the stance phase of one leg of the subject, a vertical displacement of the waist of the subject in the swing phase of one leg, an angle of the knee joint of one leg in the stance phase, and an angle of the ankle joint of one foot in the swing phase.

In the present embodiment, the walking parameter detection unit 112 detects, from walking data, the vertical displacement of the waist of the subject in the stance phase of one leg of the subject. The walking parameter detection unit 112 detects the vertical displacement of the waist of the subject in the stance phase of one leg of the subject from the time series skeleton data corresponding to the one walking cycle having been clipped. As shown in FIG. 2, a vertical displacement a of the waist is the vertical displacement of the feature point 209 indicating the waist.

In particular, the walking parameter detection unit 112 detects time series data of the vertical displacement of the waist in a predetermined period of the stance phase of one leg. More specifically, the predetermined period is a period of 1% to 60% of one walking cycle. In addition, the predetermined period may be a period of 9% to 19% of one walking cycle. The walking parameter detection unit 112 calculates, as a walking parameter, a mean value of time series data of the vertical displacement of the waist in a predetermined period of the stance phase of one leg.

It is to be noted that detection of the vertical displacement of the waist of the subject in the swing phase of one leg of the subject, the angle of the knee joint of one leg of the subject in the stance phase, and the angle of the ankle joint of one foot in the swing phase will be described in modifications of the present embodiment.

The fall risk determination unit 113 determines the fall risk of the subject using at least one of the vertical displacement of the waist in the stance phase, the vertical displacement of the waist in the swing phase, the angle of the knee joint in the stance phase, and the angle of the ankle joint in the swing phase.

In the present embodiment, the fall risk determination unit 113 determines the fall risk of the subject using the mean value of time series data of the vertical displacement of the waist.

In addition, the fall risk determination unit 113 determines whether or not the subject has a fall risk by inputting at least one of the vertical displacement of the waist in the stance phase, the vertical displacement of the waist in the swing phase, the angle of the knee joint in the stance phase, and the angle of the ankle joint in the swing phase that have been detected into a prediction model generated with at least one of the vertical displacement of the waist in the stance phase, the vertical displacement of the waist in the swing phase, the angle of the knee joint in the stance phase, and the angle of the ankle joint in the swing phase as an input value and with whether or not the subject has a fall risk as an output value.

In the present embodiment, the fall risk determination unit 113 determines whether or not the subject has a fall risk by inputting the vertical displacement of the waist that has been detected by the walking parameter detection unit 112 into a prediction model generated with the vertical displacement of the waist as an input value, and with whether or not the subject has a fall risk as an output value.

In addition, determination of fall risk of the subject using the vertical displacement of the waist in the swing phase, the angle of the knee joint in the stance phase, and the angle of the ankle joint in the swing phase will be described in modifications of the present embodiment.

The memory 12 stores in advance a prediction model generated with the vertical displacement of the waist as an input value and with whether or not the subject has a fall risk as an output value. The prediction model is a regression model with whether or not the subject has a fall risk as an objective variable, and with the time series data of the vertical displacement of the waist in the stance phase of one walking cycle as an explanatory variable. The prediction model outputs either a value indicating that the subject has a fall risk (for example, 1) or a value indicating that the subject does not have a fall risk (for example, 0).

In particular, the fall risk determination unit 113 determines the fall risk of the subject using the mean value of the time series data of the vertical displacement of the waist in the stance phase of one leg. More specifically, the fall risk determination unit 113 determines the fall risk of the subject using the mean value of the time series data of the vertical displacement of the waist in the period of 1% to 60% of one walking cycle. In addition, the fall risk determination unit 113 may determine the fall risk of the subject using the mean value of the time series data of the vertical displacement of the waist in the period of 9% to 19% of one walking cycle.

It is to be noted that the prediction model may be generated by machine learning. The machine learning includes, for example, supervised learning for learning the relationship between input and output by using training data in which a label (output information) is given to input information, unsupervised learning for constructing a structure of data only from an unlabeled input, semi-supervised learning for handling both the labeled and the unlabeled, and reinforcement learning for learning, on a trial-and-error basis, a behavior that maximizes reward. Specific methods of machine learning include a neural network (including deep learning using a multilayer neural network), genetic programming, a decision tree, a Bayesian network, and support vector machine (SVM). In the machine learning of the present disclosure, any of the above specific examples may be used.

In addition, the prediction model may output a value indicating a fall risk level. The value indicating the fall risk level is represented by 0.0 to 1.0, for example. In that case, for example, the fall risk determination unit 113 may determine that the subject does not have a fall risk when the value indicating the fall risk level is equal to or less than 0.5, and determine that the subject has a fall risk when the value indicating the fall risk level is larger than 0.5.

The evaluation result presentation unit 114 presents the evaluation result of the fall risk determined by the fall risk determination unit 113. The evaluation result presentation unit 114 outputs to the display unit 3 the evaluation result determined by the fall risk determination unit 113. The evaluation result is at least one of information indicating whether or not the subject has a fall risk determined by the fall risk determination unit 113 and an evaluation message.

The display unit 3 displays the evaluation result having been output from the evaluation result presentation unit 114. The display unit 3 is, for example, a liquid crystal display panel or a light emitting element.

It is to be noted that in order to compare the value indicating the currently determined fall risk level with the value indicating a past fall risk level, the display unit 3 may display a graph of transition of the value indicating the fall risk level. It is to be noted that the value indicating the past fall risk level is stored in the memory 12 and is read from the memory 12.

It is to be noted that the fall risk evaluation device 1 may include the camera 2 and the display unit 3. The fall risk evaluation device 1 may include the display unit 3. The fall risk evaluation device 1 may be a personal computer or a server.

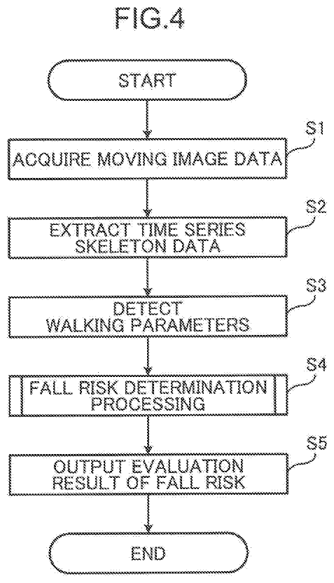

Next, the fall risk evaluation processing in the present embodiment will be described with reference to FIG. 4.

FIG. 4 is a flowchart for explaining the fall risk evaluation processing using the walking motion of a subject in the present embodiment. The flowchart shown in FIG. 4 shows a procedure of evaluation of the fall risk using the fall risk evaluation device 1.

The subject walks in front of the camera 2. The camera 2 captures an image of the walking subject. The camera 2 transmits moving image data of the walking subject to the fall risk evaluation device 1.

First, in step S1, the data acquisition unit 111 acquires the moving image data transmitted by the camera 2.

Next, in step S2, the walking parameter detection unit 112 extracts time series skeleton data from the moving image data.

Next, in step S3, the walking parameter detection unit 112 detects a walking parameter for determining the fall risk from the time series skeleton data. Here, the walking parameter in the present embodiment is a mean value of time series data of the vertical displacement of the waist of the subject in a predetermined period of the stance phase of one walking cycle. The predetermined period is a period of 1% to 60% of one walking cycle, for example. A decision method of the walking parameter will be described later.

Next, in step S4, the fall risk determination unit 113 executes the fall risk determination processing for determining the fall risk of the subject using the walking parameter. It is to be noted that the fall risk determination processing will be described later.

Next, in step S5, the evaluation result presentation unit 114 outputs to the display unit 3 the evaluation result of the fall risk determined by the fall risk determination unit 113. The evaluation result of the fall risk indicates whether or not the subject has a fall risk. It is to be noted that the evaluation result presentation unit 114 may output to the display unit 3 not only the presence or absence of a fall risk but also an evaluation message associated with the presence or absence of the fall risk. The display unit 3 displays the evaluation result of the fall risk having been output from the evaluation result presentation unit 114.

The fall risk determination processing in step S4 of FIG. 4 will now be described.

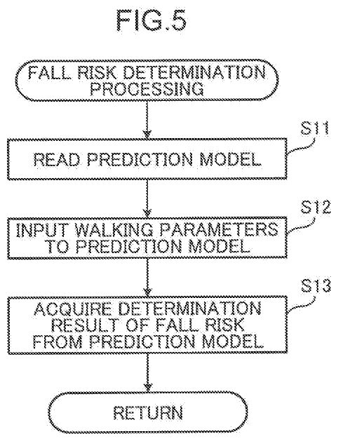

FIG. 5 is a flowchart for explaining the fall risk determination processing in step S4 of FIG. 4.

First, in step S1, the fall risk determination unit 113 reads the prediction model from the memory 12.

Next, in step S12, the fall risk determination unit 113 inputs to the prediction model the walking parameter detected by the walking parameter detection unit 112. The walking parameter in the present embodiment is a mean value of the time series data of the vertical displacement of the waist of the subject in the period of 1% to 60% of one walking cycle. The fall risk determination unit 113 inputs to the prediction model the mean value of the time series data of the vertical displacement of the waist of the subject in the period of 1% to 60% of one walking cycle.

Next, in step S13, the fall risk determination unit 113 acquires the determination result of a fall risk from the prediction model. The fall risk determination unit 113 acquires whether or not the subject has a fall risk from the prediction model as a determination result.

It is to be noted that in the fall risk determination processing of the present embodiment, by inputting a walking parameter to a prediction model generated in advance, the presence or absence of a fall risk is determined. However, the present disclosure is not particularly limited thereto. In another example of the fall risk determination processing of the present embodiment, the presence or absence of a fall risk may be determined by comparing a threshold value stored in advance with a walking parameter.

In this case, the memory 12 stores in advance a threshold value for determining whether or not the subject has a fall risk.

In addition, the fall risk determination unit 113 may determine that the subject has the fall risk when the vertical displacement of the waist in the stance phase is smaller than the threshold value, when the vertical displacement of the waist in the swing phase is smaller than the threshold value, when the angle of the knee joint in the stance phase is smaller than the threshold value, or when the angle of the ankle joint in the swing phase is smaller than the threshold value.

In the present embodiment, the fall risk determination unit 113 may determine that the subject has a fall risk when the vertical displacement of the waist is smaller than the threshold value. The fall risk determination unit 113 determines whether or not the mean value of the time series data of the vertical displacement of the waist of the subject in the period of 1% to 60% of one walking cycle is smaller than the threshold value. The fall risk determination unit 113 determines that the subject has a fall risk when the mean value of time series data of the vertical displacement of the waist of the subject in the period of 1% to 60% of one walking cycle is smaller than the threshold value. On the other hand, the fall risk determination unit 113 determines that the subject does not have a fall risk, i.e., the subject is a healthy subject when the mean value of the time series data of the vertical displacement of the waist of the subject in the period of 1% to 60% of one walking cycle is equal to or larger than the threshold value.

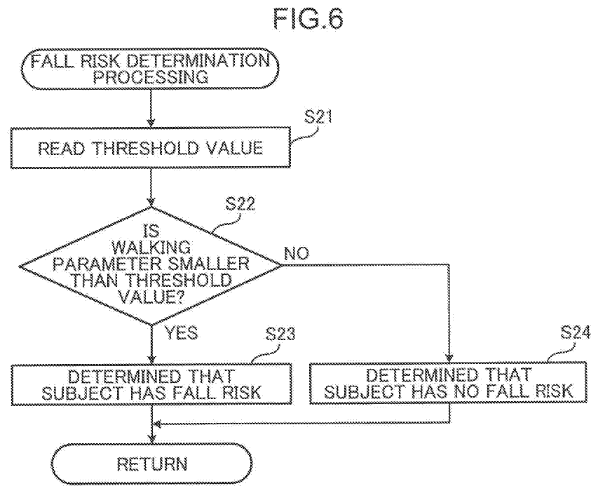

FIG. 6 is a flowchart for explaining another example of the fall risk determination processing in step S4 of FIG. 4.

First, in step S21, the fall risk determination unit 113 reads the threshold value from the memory 12.

Next, in step S22, the fall risk determination unit 113 determines whether or not the walking parameter detected by the walking parameter detection unit 112 is smaller than the threshold value. The walking parameter in the present embodiment is a mean value of the time series data of the vertical displacement of the waist of the subject in the period of 1% to 60% of one walking cycle. The fall risk determination unit 113 determines whether or not the mean value of the time series data of the vertical displacement of the waist of the subject in the period of 1% to 60% of one walking cycle is smaller than the threshold value.

Here, when it is determined that the walking parameter is smaller than the threshold value (YES in step S22), the fall risk determination unit 113 determines in step S23 that the subject has a fall risk.

On the other hand, when it is determined that the walking parameter is equal to or larger than the threshold value (NO in step S22), the fall risk determination unit 113 determines in step S24 that the subject does not have a fall risk, i.e., the subject is a healthy subject.

Thus, in the present embodiment, the vertical displacement of the waist in the stance phase of a walking subject is a parameter correlated with the presence or absence of a fall risk of the subject. Walking motion of subjects who have a fall risk tends to be different from walking motion of subjects who do not have a fall risk. Therefore, the presence or absence of the fall risk of the subject is determined by using a parameter correlated with the presence or absence of the fall risk of the walking subject, and thus the fall risk of the subject can be evaluated with high accuracy.

Furthermore, the vertical displacement of the waist in the stance phase of a walking subject can be easily detected from image data obtained by capturing an image of the walking subject, for example, and hence a large-scale device is unnecessary. Therefore, this configuration can easily evaluate the fall risk of a subject.

The walking parameters and the prediction models in the present embodiment are decided by experiments. Hereinafter, a decision method of a walking parameter and a prediction model in the present embodiment will be described.

The total number of subjects who participated in the experiment was 92. There were 27 male subjects and 65 female subjects. Based on the results of previous studies, the determination criterion for the presence or absence of the fall risk is whether or not a person can hold a state of standing on one foot with his eyes open for 30 seconds. The subjects were asked to hold the state of standing on one foot with their eyes open, and the holding time was measured. It was determined that there was a fall risk when the holding time was equal to or less than 30 seconds, and it was determined that there was no fall risk when the holding time was longer than 30 seconds. As a result of the determination, the subjects included 34 subjects who had a fall risk. Of the subjects who had a fall risk, 10 were male and 24 were female. In the experiment, the subjects performed walking in front of the camera. Images of the walking subjects were captured by the camera, and the skeleton data of each subject was extracted from the moving image data. Then, time series data of the vertical displacement of the waist of each subject was detected from the extracted skeleton data.

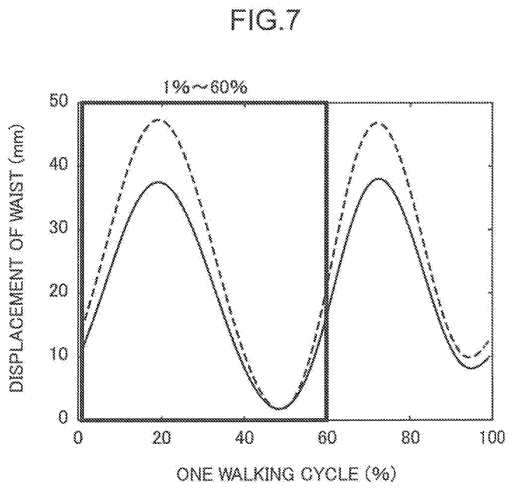

FIG. 7 is a view showing a change in the vertical displacement of the waist in one walking cycle in the present embodiment. In FIG. 7, the vertical axis represents the vertical displacement of the waist, and the horizontal axis represents one normalized walking cycle. In addition, in FIG. 7, the dashed line represents an average waveform of the vertical displacements of the waist of the subjects who do not have a fall risk, and the solid line represents an average waveform of the vertical displacements of the waist of the subjects who have a fall risk.

In the experiment, one normalized walking cycle was divided into ten intervals, and the mean value of the vertical displacements of the waist in one interval or two or more consecutive intervals was calculated for each subject. Then, a plurality of prediction models was created with whether or not the subject has a fall risk as an objective variable, and with the mean value of the vertical displacements of the waist in one interval or two or more consecutive intervals as an explanatory variable. The plurality of prediction models was evaluated by cross validation. Leave-one-out cross validation was adopted as the cross validation. Then, a receiver operating characteristic (ROC) curve of each of the plurality of prediction models was calculated. Furthermore, an area under curve (AUC) value of the ROC curve of each of the plurality of prediction models was calculated, and the prediction model with the highest AUC value was selected.

In the present embodiment, the prediction model created with the mean value of the vertical displacements of the waist in the period of 1% to 60% of one walking cycle as the explanatory variable had the highest AUC value.

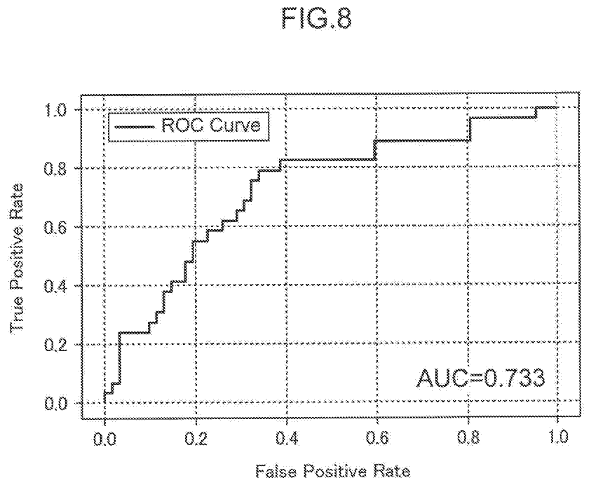

FIG. 8 is a view showing an ROC curve obtained as a result of determining the presence or absence of a fall risk using a prediction model in the present embodiment.

The prediction model in the present embodiment was created with whether or not the subject has a fall risk as an objective variable, and with the mean value of the vertical displacements of the waist in the period of 1% to 60% of one walking cycle as an explanatory variable. In FIG. 8, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 8 was obtained by plotting the true positive rate and the false positive rate of the prediction model created with the mean value of the vertical displacements of the waist in the period of 1% to 60% of one walking cycle as an explanatory variable. The AUC value of the ROC curve shown in FIG. 8 was 0.733. The AUC value is the area below the ROC curve. It is true that the larger the AUC value is (the more it approaches 1), the higher the performance of the prediction model is. In this case, the mean value of the vertical displacements of the waist in the period of 1% to 60% of one walking cycle is determined as a walking parameter. In addition, the prediction model created with the mean value of the vertical displacements of the waist in the period of 1% to 60% of one walking cycle as an explanatory variable is determined as a prediction model to be used by the fall risk determination unit 113.

The memory 12 stores in advance a prediction model generated with the mean value of time series data of the vertical displacement of the waist in the period of 1% to 60% of one walking cycle as an input value and with whether or not the subject has a fall risk as an output value. The walking parameter detection unit 112 detects time series data of the vertical displacement of the waist in the period of 1% to 60% of one walking cycle. By inputting the mean value of time series data of the vertical displacement of the waist in the period of 1% to 60% of one walking cycle into the prediction model, the fall risk determination unit 113 acquires, from the prediction model, a determination result indicating whether or not the subject has a fall risk.

In addition, in the period of 1% to 60% of one walking cycle shown in FIG. 7, the average waveform of the vertical displacements of the waist of the subjects who have a fall risk is smaller than the average waveform of the vertical displacements of the waist of the subjects who do not have a fall risk. Therefore, a value between an average of the mean values of time series data of the vertical displacement of the waist in the period of 1% to 60% of one walking cycle of the subjects who have a fall risk and an average of the mean values of time series data of the vertical displacement of the waist in the period of 1% to 60% of one walking cycle of the subjects who do not have a fall risk, having been experimentally obtained, may be stored in the memory 12 as the threshold value. The fall risk determination unit 113 may determine the fall risk by comparing the mean value of time series data of the vertical displacement of the waist of the subject in the period of 1% to 60% of one walking cycle with the threshold value stored in advance.

It is to be noted that while in the present embodiment, the walking parameter is a mean value of time series data of the vertical displacement of the waist in the period of 1% to 60% of one walking cycle, the present disclosure is not particularly limited thereto. Various examples of the walking parameters of the present embodiment will be described below.

First, the walking parameters in the first modification of the present embodiment will be described.

The walking parameter in the first modification of the present embodiment may be a mean value of time series data of the vertical displacement of the waist in the period of 9% to 19% of one walking cycle.

In the first modification of the present embodiment, similar to the above experiment, time series data of the vertical displacement of the waist of each of the plurality of subjects was detected. In addition, a prediction model was created with whether or not the subject has a fall risk as an objective variable and with the mean value of the vertical displacements of the waist in the period of 9% to 19% of one walking cycle as an explanatory variable. The prediction model was evaluated by cross validation. Leave-one-out cross validation was adopted as the cross validation. Then, the ROC curve of the prediction model was calculated. Furthermore, the AUC value of the ROC curve of the prediction model was calculated.

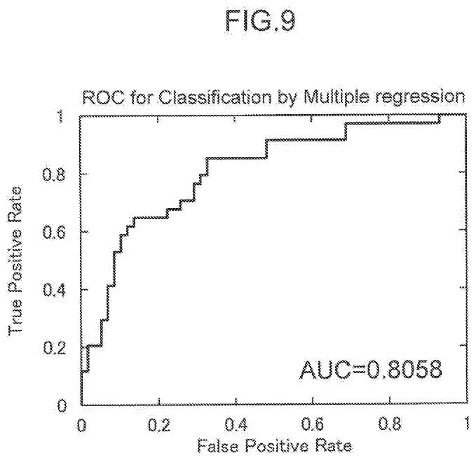

FIG. 9 is a view showing an ROC curve obtained as a result of determining the presence or absence of a fall risk using a prediction model in a first modification of the present embodiment.

The prediction model in the first modification of the present embodiment was created with whether or not the subject has a fall risk as an objective variable, and with the mean value of the vertical displacements of the waist in the period of 9% to 19% of one walking cycle as an explanatory variable. In FIG. 9, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 9 was obtained by plotting the true positive rate and the false positive rate of the prediction model created with the mean value of the vertical displacements of the waist in the period of 9% to 19% of one walking cycle as an explanatory variable. The AUC value of the ROC curve shown in FIG. 9 was 0.8058. In this case, the mean value of the vertical displacements of the waist in the period of 9% to 19% of one walking cycle is determined as a walking parameter. In addition, the prediction model created with the mean value of the vertical displacements of the waist in the period of 9% to 19% of one walking cycle as the explanatory variable is determined as the prediction model used by the fall risk determination unit 113.

The memory 12 stores in advance a prediction model generated with the mean value of time series data of the vertical displacement of the waist in the period of 9% to 19% of one walking cycle as an input value and with whether or not the subject has a fall risk as an output value.

The walking parameter detection unit 112 detects time series data of the vertical displacement of the waist in the period of 9% to 19% of one walking cycle. In addition, the walking parameter detection unit 112 calculates the mean value of time series data of the vertical displacement of the waist in the period of 9% to 19% of one walking cycle.

The fall risk determination unit 113 determines the presence or absence of a fall risk of the subject by using the mean value of time series data of the vertical displacement of the waist in the period of 9% to 19% of one walking cycle. By inputting the mean value of time series data of the vertical displacement of the waist in the period of 9% to 19% of one walking cycle into the prediction model, the fall risk determination unit 113 acquires, from the prediction model, a determination result indicating whether or not the subject has a fall risk.

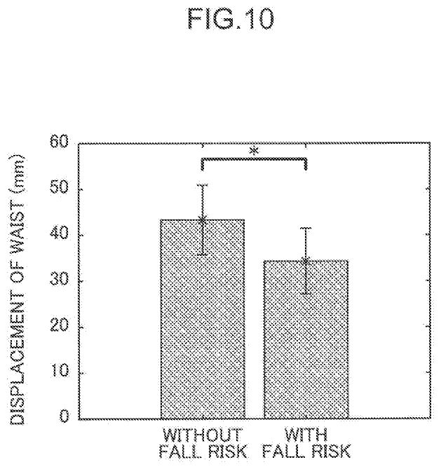

FIG. 10 is a view showing an average of mean values of time series data of the vertical displacement of the waist of the subjects who do not have a fall risk in the period of 9% to 19% of one walking cycle and an average of mean values of time series data of the vertical displacement of the waist of the subjects who have a fall risk in the period of 9% to 19% of one walking cycle in the first modification of the present embodiment.

As shown in FIG. 10, the average of mean values of time series data of the vertical displacement of the waist of the subjects who do not have a fall risk in the period of 9% to 19% of one walking cycle was 43.3 mm, and the average of mean values of time series data of the vertical displacement of the waist of the subjects who have a fall risk in the period of 9% to 19% of one walking cycle was 34.3 mm.

Thus, in the period of 9% to 19% of one walking cycle, the average of the mean values of time series data of the vertical displacement of the waist of the subjects who have a fall risk is smaller than the average of the mean values of time series data of the vertical displacement of the waist of the subjects who do not have a fall risk. Therefore, a value between an average of the mean values of time series data of the vertical displacement of the waist in the period of 9% to 19% of one walking cycle of the subjects who have a fall risk and an average of the mean values of time series data of the vertical displacement of the waist in the period of 9% to 19% of one walking cycle of the subjects who do not have a fall risk, having been experimentally obtained, may be stored in the memory 12 as the threshold value. The fall risk determination unit 113 may determine the presence or absence of a fall risk by comparing the mean value of time series data of the vertical displacement of the waist of the subject in the period of 9% to 19% of one walking cycle with the threshold value stored in advance.

Subsequently, the walking parameters in the second modification of the present embodiment will be described.

The walking parameter in the second modification of the present embodiment may be a mean value of time series data of the vertical displacement of the waist of the subject in the swing phase of one leg of the subject.

In the second modification of the present embodiment, similar to the above experiment, time series data of the vertical displacement of the waist of each of the plurality of subjects was detected from the skeleton data of the plurality of subjects including a subject who does not have a fall risk and a subject who has a fall risk.

In the second modification of the present embodiment, similar to the above experiment, time series data of the vertical displacement of the waist of each of the plurality of subjects was detected. In addition, a prediction model was created with whether or not the subject has a fall risk as an objective variable and with the mean value of the vertical displacements of the waist in a predetermined period of the swing phase as an explanatory variable. The predetermined period is a period of 61% to 100% of one walking cycle. The prediction model was evaluated by cross validation. Leave-one-out cross validation was adopted as the cross validation. Then, the ROC curve of the prediction model was calculated. Furthermore, the AUC value of the ROC curve of the prediction model was calculated.

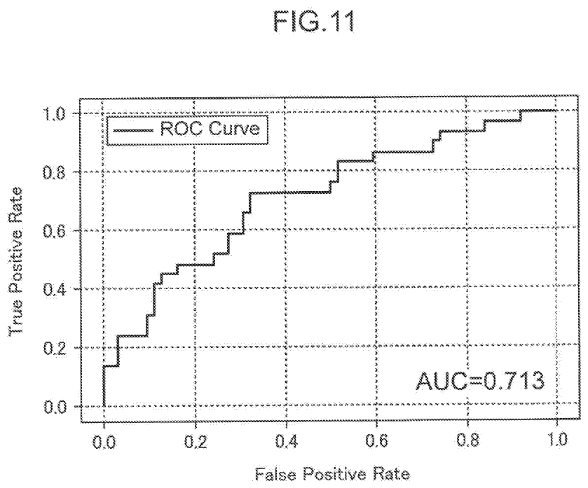

FIG. 11 is a view showing an ROC curve obtained as a result of determining the presence or absence of a fall risk using a prediction model in the second modification of the present embodiment.

The prediction model in the second modification of the present embodiment was created with whether or not the subject has a fall risk as an objective variable, and with the mean value of the vertical displacements of the waist in the period of 61% to 100% of one walking cycle as an explanatory variable. In FIG. 11, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 11 was obtained by plotting the true positive rate and the false positive rate of the prediction model created with the mean value of the vertical displacements of the waist in the period of 61% to 100% of one walking cycle as an explanatory variable. The AUC value of the ROC curve shown in FIG. 11 was 0.713. In this case, the mean value of the vertical displacements of the waist in the period of 61% to 1000% of one walking cycle is determined as a walking parameter. In addition, the prediction model created with the mean value of the vertical displacements of the waist in the period of 61% to 100% of one walking cycle as the explanatory variable is determined as the prediction model used by the fall risk determination unit 113.

The walking parameter detection unit 112 detects, from walking data, the vertical displacement of the waist of the subject. The walking parameter detection unit 112 detects the vertical displacement of the waist of the subject from the time series skeleton data corresponding to the one walking cycle having been clipped. In particular, the walking parameter detection unit 112 detects time series data of the vertical displacement of the waist in a predetermined period of the swing phase of one leg. More specifically, the predetermined period is a period of 61% to 100% of one walking cycle. The walking parameter detection unit 112 detects time series data of the vertical displacement of the waist in the period of 61% to 100% of one walking cycle. In addition, the walking parameter detection unit 112 calculates the mean value of time series data of the vertical displacement of the waist in the period of 61% to 100% of one walking cycle.

The memory 12 stores in advance a prediction model generated with the vertical displacement of the waist in a predetermined period of the swing phase as an input value and with whether or not the subject has a fall risk as an output value. The prediction model is a regression model with whether or not the subject has a fall risk as an objective variable, and with the time series data of the vertical displacement of the waist of one walking cycle as an explanatory variable. In particular, the memory 12 stores in advance a prediction model generated with the mean value of time series data of the vertical displacement of the waist in the period of 61% to 100% of one walking cycle as an input value and with whether or not the subject has a fall risk as an output value.

The fall risk determination unit 113 determines the presence or absence of a fall risk of the subject by using the mean value of time series data of the vertical displacement of the waist of a predetermined period in the swing phase. The fall risk determination unit 113 determines whether or not the subject has a fall risk by inputting the mean value of time series data of the vertical displacement of the waist that has been detected by the walking parameter detection unit 112 into a prediction model generated with the mean value of time series data of the vertical displacement of the waist in a predetermined period of the swing phase as an input value, and with whether or not the subject has a fall risk as an output value.

In addition, the fall risk determination unit 113 determines the presence or absence of a fall risk of the subject by using the mean value of time series data of the vertical displacement of the waist in a predetermined period in the swing phase of one leg. More specifically, the fall risk determination unit 113 determines the presence or absence of a fall risk of the subject by using the mean value of time series data of the vertical displacement of the waist in the period of 61% to 100% of one walking cycle. By inputting the mean value of time series data of the vertical displacement of the waist in the period of 61% to 100% of one walking cycle into the prediction model, the fall risk determination unit 113 acquires, from the prediction model, a determination result indicating whether or not the subject has a fall risk.

In addition, in the period of 61% to 100% of one walking cycle shown in FIG. 7, the average waveform of the vertical displacements of the waist of the subjects who have a fall risk is smaller than the average waveform of the vertical displacements of the waist of the subjects who do not have a fall risk. Therefore, a value between an average of the mean values of time series data of the vertical displacement of the waist in the period of 61% to 100% of one walking cycle of the subjects who have a fall risk and an average of the mean values of time series data of the vertical displacement of the waist in the period of 61% to 100% of one walking cycle of the subjects who do not have a fall risk, having been experimentally obtained, may be stored in the memory 12 as the threshold value. The fall risk determination unit 113 may determine the presence or absence of a fall risk by comparing the mean value of time series data of the vertical displacement of the waist of the subject in the period of 61% to 100% of one walking cycle with the threshold value stored in advance.

Subsequently, the walking parameters in the third modification of the present embodiment will be described.

The walking parameter in the third modification of the present embodiment may be a mean value of time series data of the angle of the knee joint of one leg in a predetermined period of the stance phase of one leg.

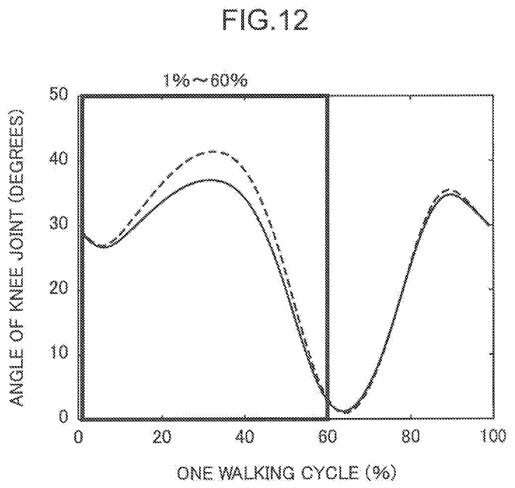

FIG. 12 is a view showing a change in the angle of one knee joint in one walking cycle in the third modification of the present embodiment. In FIG. 12, the vertical axis represents the angle of the knee joint, and the horizontal axis represents one normalized walking cycle. In addition, in FIG. 12, the dashed line represents an average waveform of the angles of one knee joint of the subjects who do not have a fall risk, and the solid line represents an average waveform of the angles of one knee joint of the subjects who have a fall risk.

In the third modification of the present embodiment, similar to the above experiment, time series data of the angle of one knee joint of each of the plurality of subjects was detected from the skeleton data of the plurality of subjects including a subject who does not have a fall risk and a subject who has a fall risk. As shown in FIG. 2, an angle γ of the knee joint is an angle formed in the sagittal plane by a straight line connecting the feature point 211 indicating the right knee joint and the feature point 210 indicating the right hip joint and a straight line connecting the feature point 211 indicating the right knee joint and the feature point 212 indicating the right ankle joint.

In the experiment, one normalized walking cycle was divided into ten intervals, and the mean value of the angles of one knee joint in one interval or two or more consecutive intervals was calculated for each subject. Then, a plurality of prediction models was created with whether or not the subject has a fall risk as an objective variable, and with the mean value of the angles of one knee joint in one interval or two or more consecutive intervals as an explanatory variable. The plurality of prediction models was evaluated by cross validation. Leave-one-out cross validation was adopted as the cross validation. Then, the ROC curve of each of the plurality of prediction models was calculated. Furthermore, the AUC value of the ROC curve of each of the plurality of prediction models was calculated, and the prediction model with the highest AUC value was selected.

In the third modification of the present embodiment, the prediction model created with the mean value of the angles of one knee joint in the period of 1% to 60% of one walking cycle as the explanatory variable had the highest AUC value.

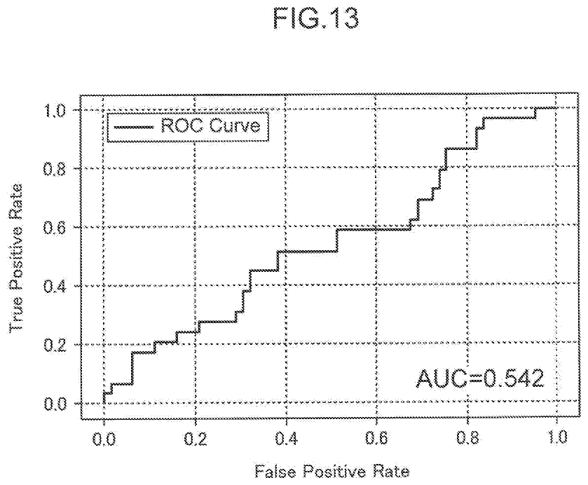

FIG. 13 is a view showing an ROC curve obtained as a result of determining the presence or absence of a fall risk using a prediction model in the third modification of the present embodiment.

The prediction model in the third modification of the present embodiment was created with whether or not the subject has a fall risk as an objective variable, and with the mean value of the angles of one knee joint in the period of 1% to 60% of one walking cycle as an explanatory variable. In FIG. 13, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 13 was obtained by plotting the true positive rate and the false positive rate of the prediction model created with the mean value of the angles of the knee joint in the period of 1% to 60% of one walking cycle as an explanatory variable. The AUC value of the ROC curve shown in FIG. 13 was 0.542. In this case, the mean value of the angles of the knee joint in the period of 1% to 60% of one walking cycle is determined as a walking parameter. In addition, the prediction model created with the mean value of the angles of the knee joint in the period of 1% to 60% of one walking cycle as an explanatory variable is determined as a prediction model to be used by the fall risk determination unit 113.

The walking parameter detection unit 112 detects, from walking data, the angle of the knee joint of one leg of the subject. The walking parameter detection unit 112 detects the angle of the knee joint of one leg of the subject from the time series skeleton data corresponding to the one walking cycle having been clipped. In particular, the walking parameter detection unit 112 detects time series data of the angle of the knee joint in a predetermined period of the stance phase of one leg. More specifically, the predetermined period is a period of 1% to 60% of one walking cycle. The walking parameter detection unit 112 detects time series data of the angle of the knee joint of one leg in a period of 1% to 60% of one walking cycle. In addition, the walking parameter detection unit 112 calculates the mean value of time series data of the angle of the knee joint of one leg in a period of 1% to 60% of one walking cycle.

It is to be noted that in the third modification of the present embodiment, since the one walking cycle is a period from when the right foot of the subject touches the ground to when the right foot of the subject touches the ground again, the walking parameter detection unit 112 detects the angle γ of the knee joint of the right leg. Ina case where one walking cycle is a period from when the left foot of the subject touches the ground to when the left foot touches the ground again, the walking parameter detection unit 112 may detect the angle γ of the knee joint of the left leg.

The memory 12 stores in advance a prediction model generated with the angle of the knee joint as an input value and with whether or not the subject has a fall risk as an output value. The prediction model is a regression model with whether or not the subject has a fall risk as an objective variable, and with the time series data of the angle of the knee joint of one walking cycle as an explanatory variable. In particular, the memory 12 stores in advance a prediction model generated with the mean value of time series data of the angle of the knee joint of one leg in the period of 1% to 60% of one walking cycle as an input value and with whether or not the subject has a fall risk as an output value.

The fall risk determination unit 113 determines the presence or absence of a fall risk of the subject by using the angle of the knee joint. The fall risk determination unit 113 determines whether or not the subject has a fall risk by inputting the angle of the knee joint that has been detected by the walking parameter detection unit 112 into a prediction model generated with the angle of the knee joint as an input value, and with whether or not the subject has a fall risk as an output value.

In addition, the fall risk determination unit 113 determines the presence or absence of a fall risk of the subject by using the mean value of time series data of the angle of the knee joint in a predetermined period in the stance phase of one leg. More specifically, the fall risk determination unit 113 determines the presence or absence of a fall risk of the subject by using the mean value of time series data of the angle of the knee joint of one leg in the period of 1% to 60% of one walking cycle. By inputting the mean value of time series data of the angle of the knee joint of one leg in the period of 1% to 60% of one walking cycle into the prediction model, the fall risk determination unit 113 acquires, from the prediction model, a determination result indicating whether or not the subject has a fall risk.

In addition, in the period of 1% to 60% of one walking cycle shown in FIG. 12, the average waveform of the angles of the knee joint of one leg of the subjects who have a fall risk is smaller than the average waveform of the angles of the knee joint of one leg of the subjects who do not have a fall risk. Therefore, a value between an average of the mean values of time series data of the angle of the knee joint of one leg in the period of 1% to 60% of one walking cycle of the subjects who have a fall risk and an average of the mean values of time series data of the angle of the knee joint of one leg in the period of 1% to 60% of one walking cycle of the subjects who do not have a fall risk, having been experimentally obtained, may be stored in the memory 12 as the threshold value. The fall risk determination unit 113 may determine the presence or absence of a fall risk by comparing the mean value of time series data of the angle of the knee joint of one leg of the subject in the period of 1% to 60% of one walking cycle with the threshold value stored in advance.

Subsequently, the walking parameters in the fourth modification of the present embodiment will be described.

The walking parameter in the fourth modification of the present embodiment may be an angle of the knee joint of one leg at a predetermined time point of the stance phase of one leg.

In the fourth modification of the present embodiment, similar to the above experiment, time series data of the angle of one knee joint of each of the plurality of subjects was detected from the skeleton data of the plurality of subjects including a subject who does not have a fall risk and a subject who has a fall risk. In addition, a prediction model was created with whether or not the subject has a fall risk as an objective variable and with the angle of one knee joint at the time point of 35% of one walking cycle as an explanatory variable. The prediction model was evaluated by cross validation. Leave-one-out cross validation was adopted as the cross validation. Then, the ROC curve of the prediction model was calculated. Furthermore, the AUC value of the ROC curve of the prediction model was calculated.

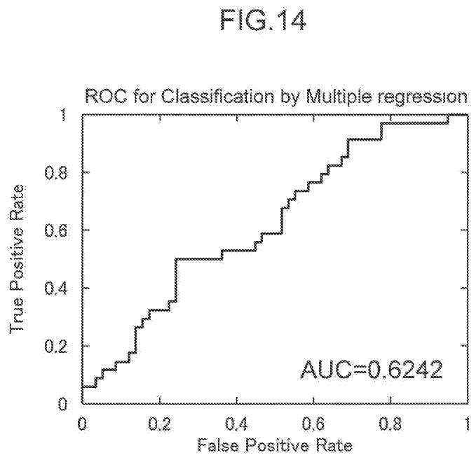

FIG. 14 is a view showing an ROC curve obtained as a result of determining the presence or absence of a fall risk using a prediction model in the fourth modification of the present embodiment.

The prediction model in the fourth modification of the present embodiment was created with whether or not the subject has a fall risk as an objective variable, and with the angle of one knee joint at the time point of 35% of one walking cycle as an explanatory variable. In FIG. 14, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 14 was obtained by plotting the true positive rate and the false positive rate of the prediction model created with the angle of the knee joint at the time point of 35% of one walking cycle as an explanatory variable. The AUC value of the ROC curve shown in FIG. 14 was 0.6242. In this case, the angle of the knee joint at the time point of 35% of one walking cycle is determined as a walking parameter. In addition, the prediction model created with the angle of the knee joint at the time point of 35% of one walking cycle as an explanatory variable is determined as a prediction model to be used by the fall risk determination unit 113.

The walking parameter detection unit 112 detects, from walking data, the angle of the knee joint of one leg of the subject. The walking parameter detection unit 112 detects the angle of the knee joint of one leg of the subject from the time series skeleton data corresponding to the one walking cycle having been clipped. In particular, the walking parameter detection unit 112 detects the angle of the knee joint in a predetermined period of the stance phase of one leg. More specifically, the walking parameter detection unit 112 detects the angle of the knee joint of one leg at the time point of 35% of one walking cycle.

The memory 12 stores in advance a prediction model generated with the angle of the knee joint as an input value and with whether or not the subject has a fall risk as an output value. The prediction model is a regression model with whether or not the subject has a fall risk as an objective variable, and with the time series data of the angle of the knee joint of one walking cycle as an explanatory variable. In particular, the memory 12 stores in advance a prediction model generated with the angle of the knee joint of one leg at the time point of 35% of one walking cycle as an input value and with whether or not the subject has a fall risk as an output value.

The fall risk determination unit 113 determines the presence or absence of a fall risk of the subject by using the angle of the knee joint. The fall risk determination unit 113 determines whether or not the subject has a fall risk by inputting the angle of the knee joint that has been detected by the walking parameter detection unit 112 into a prediction model generated with the angle of the knee joint as an input value, and with whether or not the subject has a fall risk as an output value.

In addition, the fall risk determination unit 113 determines the presence or absence of a fall risk of the subject by using the mean value of time series data of the angle of the knee joint in a predetermined period in the stance phase of one leg. More specifically, the fall risk determination unit 113 determines the presence or absence of a fall risk of the subject by using the angle of the knee joint of one leg at the time point of 35% of one walking cycle. By inputting the angle of the knee joint of one leg at the time point of 35% of one walking cycle into the prediction model, the fall risk determination unit 113 acquires, from the prediction model, a determination result indicating whether or not the subject has a fall risk.

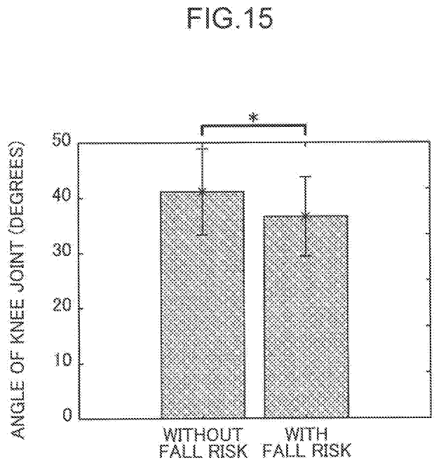

FIG. 15 is a view showing an average of the angles of the knee joint of one leg of the subjects who do not have a fall risk at the time point of 35% of one walking cycle and an average of the angles of the knee joint of one leg of the subjects who have a fall risk at the time point of 35% of one walking cycle in the fourth modification of the present embodiment.

As shown in FIG. 15, the average of the angles of the knee joint of one leg of the subjects who do not have a fall risk at the time point of 35% of one walking cycle was 41.0 degrees, and the average of the angles of the knee joint of one leg of the subjects who have a fall risk at the time point of 35% of one walking cycle was 36.6 degrees.

Thus, at the time point of 35% of one walking cycle, the average of the angles of the knee joint of one leg of the subjects who have a fall risk is smaller than the average of the angles of the knee joint of one leg of the subjects who do not have a fall risk. Therefore, a value between an average of the angles of the knee joint of one leg at the time point of 35% of one walking cycle of the subjects who have a fall risk and an average of the angles of the knee joint of one leg at the time point of 35% of one walking cycle of the subjects who do not have a fall risk, having been experimentally obtained, may be stored in the memory 12 as the threshold value. The fall risk determination unit 113 may determine the presence or absence of a fall risk by comparing the angle of the knee joint of one leg of the subject at the time point of 35% of one walking cycle with the threshold value stored in advance.

Subsequently, the walking parameters in the fifth modification of the present embodiment will be described.

The walking parameter in the fifth modification of the present embodiment may be a mean value of time series data of the angle of the ankle joint of one foot in a predetermined period of the swing phase of one leg.

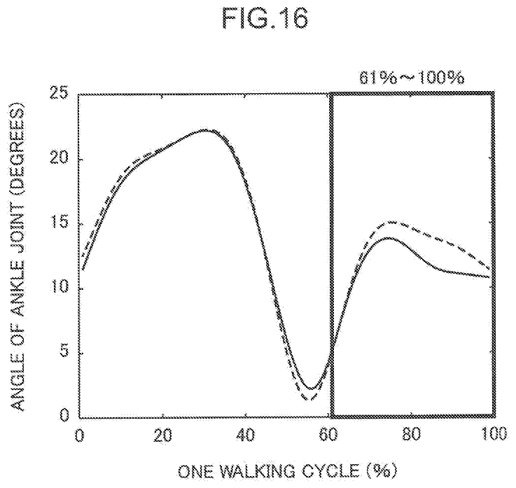

FIG. 16 is a view showing a change in the angle of one ankle joint in one walking cycle in the fifth modification of the present embodiment. In FIG. 16, the vertical axis represents the angle of the ankle joint, and the horizontal axis represents one normalized walking cycle. In addition, in FIG. 16, the dashed line represents an average waveform of the angles of one ankle joint of the subjects who do not have a fall risk, and the solid line represents an average waveform of the angles of one ankle joint of the subjects who have a fall risk.

In the fifth modification of the present embodiment, similar to the above experiment, time series data of the angle of one ankle joint of each of the plurality of subjects was detected from the skeleton data of the plurality of subjects including a subject who does not have a fall risk and a subject who has a fall risk. As shown in FIG. 2, an angle θ of the ankle joint is an angle formed in the sagittal plane by a straight line connecting the feature point 212 indicating the right ankle joint and the feature point 211 indicating the right knee joint and a straight line connecting the feature point 212 indicating the right ankle joint and the feature point 213 indicating the right toe.

In the experiment, one normalized walking cycle was divided into ten intervals, and the mean value of the angles of one ankle joint in one interval or two or more consecutive intervals was calculated for each subject. Then, a plurality of prediction models was created with whether or not the subject has a fall risk as an objective variable, and with the mean value of the angles of one ankle joint in one interval or two or more consecutive intervals as an explanatory variable. The plurality of prediction models was evaluated by cross validation. Leave-one-out cross validation was adopted as the cross validation. Then, the ROC curve of each of the plurality of prediction models was calculated. Furthermore, the AUC value of the ROC curve of each of the plurality of prediction models was calculated, and the prediction model with the highest AUC value was selected.

In the fifth modification of the present embodiment, the prediction model created with the mean value of the angles of one ankle joint in the period of 61% to 100% of one walking cycle as the explanatory variable had the highest AUC value.

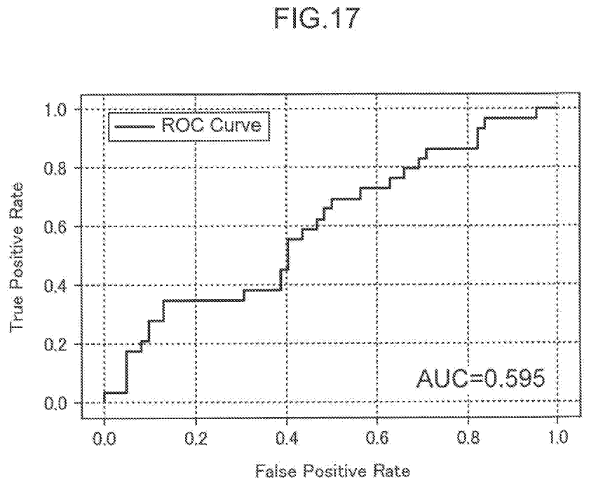

FIG. 17 is a view showing an ROC curve obtained as a result of determining the presence or absence of a fall risk using a prediction model in the fifth modification of the present embodiment.

The prediction model in the fifth modification of the present embodiment was created with whether or not the subject has a fall risk as an objective variable, and with the mean value of the angles of one ankle joint in the period of 61% to 100% of one walking cycle as an explanatory variable. In FIG. 17, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 17 was obtained by plotting the true positive rate and the false positive rate of the prediction model created with the mean value of the angles of the ankle joint in the period of 61% to 100% of one walking cycle as an explanatory variable. The AUC value of the ROC curve shown in FIG. 17 was 0.595. In this case, the mean value of the angles of the ankle joint in the period of 61% to 100% of one walking cycle is determined as a walking parameter. In addition, the prediction model created with the mean value of the angles of the ankle joint in the period of 61% to 100% of one walking cycle as an explanatory variable is determined as a prediction model to be used by the fall risk determination unit 113.

The walking parameter detection unit 112 detects, from walking data, the angle of the ankle joint of one foot of the subject. The walking parameter detection unit 112 detects the angle of the ankle joint of one foot of the subject from the time series skeleton data corresponding to the one walking cycle having been clipped. In particular, the walking parameter detection unit 112 detects time series data of the angle of the ankle joint in a predetermined period of the swing phase of one leg. More specifically, the predetermined period is a period of 61% to 100% of one walking cycle. The walking parameter detection unit 112 detects time series data of the angle of the ankle joint of one foot in a period of 61% to 100% of one walking cycle. In addition, the walking parameter detection unit 112 calculates the mean value of time series data of the angle of the ankle joint of one foot in a period of 61% to 100% of one walking cycle.

It is to be noted that in the fifth modification of the present embodiment, since the one walking cycle is a period from when the right foot of the subject touches the ground to when the right foot touches the ground again, the walking parameter detection unit 112 detects the angle θ of the ankle joint of the right foot. In a case where one walking cycle is a period from when the left foot of the subject touches the ground to when the left foot touches the ground again, the walking parameter detection unit 112 may detect the angle θ of the ankle joint of the left foot.

The memory 12 stores in advance a prediction model generated with the angle of the ankle joint as an input value and with whether or not the subject has a fall risk as an output value. The prediction model is a regression model with whether or not the subject has a fall risk as an objective variable, and with the time series data of the angle of the ankle joint of one walking cycle as an explanatory variable. In particular, the memory 12 stores in advance a prediction model generated with the mean value of time series data of the angle of the ankle joint of one foot in the period of 61% to 100% of one walking cycle as an input value and with whether or not the subject has a fall risk as an output value.

The fall risk determination unit 113 determines the presence or absence of a fall risk of the subject by using the angle of the ankle joint. The fall risk determination unit 113 determines whether or not the subject has a fall risk by inputting the angle of the ankle joint that has been detected by the walking parameter detection unit 112 into a prediction model generated with the angle of the ankle joint as an input value, and with whether or not the subject has a fall risk as an output value.

In addition, the fall risk determination unit 113 determines the presence or absence of a fall risk of the subject by using the mean value of time series data of the angle of the ankle joint in a predetermined period in the swing phase of one leg. More specifically, the fall risk determination unit 113 determines the presence or absence of a fall risk of the subject by using the mean value of time series data of the angle of the ankle joint of one foot in the period of 61% to 100% of one walking cycle. By inputting the mean value of time series data of the angle of the ankle joint of one foot in the period of 61% to 100% of one walking cycle into the prediction model, the fall risk determination unit 113 acquires, from the prediction model, a determination result indicating whether or not the subject has a fall risk.

In addition, in the period of 61% to 100% of one walking cycle shown in FIG. 16, the average waveform of the angles of the ankle joint of one foot of the subjects who have a fall risk is smaller than the average waveform of the angles of the ankle joint of one foot of the subjects who do not have a fall risk. Therefore, a value between an average of the mean values of time series data of the angle of the ankle joint of one foot in the period of 61% to 100% of one walking cycle of the subjects who have a fall risk and an average of the mean values of time series data of the angle of the ankle joint of one foot in the period of 61% to 10% of one walking cycle of the subjects who do not have a fall risk, having been experimentally obtained, may be stored in the memory 12 as the threshold value. The fall risk determination unit 113 may determine the presence or absence of a fall risk by comparing the mean value of time series data of the angle of the ankle joint of one foot of the subject in the period of 61% to 100% of one walking cycle with the threshold value stored in advance.

Subsequently, the walking parameters in the sixth modification of the present embodiment will be described.

The walking parameter in the sixth modification of the present embodiment may be a mean value of time series data of the angle of the ankle joint of one foot in the period of 84% to 89% of one walking cycle.

In the sixth modification of the present embodiment, similar to the above experiment, time series data of the angle of the ankle joint of one foot of each of the plurality of subjects was detected. In addition, a prediction model was created with whether or not the subject has a fall risk as an objective variable and with the mean value of the angles of the ankle joint of one foot in the period of 84% to 89% of one walking cycle as an explanatory variable. The prediction model was evaluated by cross validation. Leave-one-out cross validation was adopted as the cross validation. Then, the ROC curve of the prediction model was calculated. Furthermore, the AUC value of the ROC curve of the prediction model was calculated.

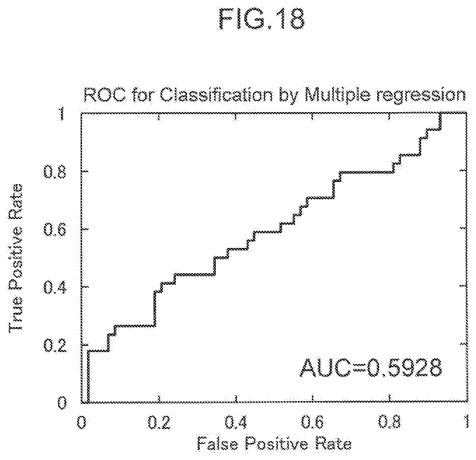

FIG. 18 is a view showing an ROC curve obtained as a result of determining the presence or absence of a fall risk using a prediction model in the sixth modification of the present embodiment.

The prediction model in the sixth modification of the present embodiment was created with whether or not the subject has a fall risk as an objective variable, and with the mean value of the angles of one ankle joint in the period of 84% to 89% of one walking cycle as an explanatory variable. In FIG. 18, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 18 was obtained by plotting the true positive rate and the false positive rate of the prediction model created with the mean value of the angles of the ankle joint in the period of 84% to 89% of one walking cycle as an explanatory variable. The AUC value of the ROC curve shown in FIG. 18 was 0.5928. In this case, the mean value of the angles of the ankle joint in the period of 84% to 89% of one walking cycle is determined as a walking parameter. In addition, the prediction model created with the mean value of the angles of the ankle joint in the period of 84% to 89% of one walking cycle as an explanatory variable is determined as a prediction model to be used by the fall risk determination unit 113.

The memory 12 stores in advance a prediction model generated with the mean value of time series data of the angle of the ankle joint of one foot in the period of 84% to 89% of one walking cycle as an input value and with whether or not the subject has a fall risk as an output value.

The walking parameter detection unit 112 detects time series data of the angle of the ankle joint of one foot in the period of 84% to 89% of one walking cycle. In addition, the walking parameter detection unit 112 calculates the mean value of time series data of the angle of the ankle joint of one foot in the period of 84% to 89% of one walking cycle.

The fall risk determination unit 113 determines the presence or absence of a fall risk of the subject by using the mean value of time series data of the angle of the ankle joint of one foot in the period of 84% to 89% of one walking cycle. By inputting the mean value of time series data of the angle of the ankle joint of one foot in the period of 84% to 89% of one walking cycle into the prediction model, the fall risk determination unit 113 acquires, from the prediction model, a determination result indicating whether or not the subject has a fall risk.

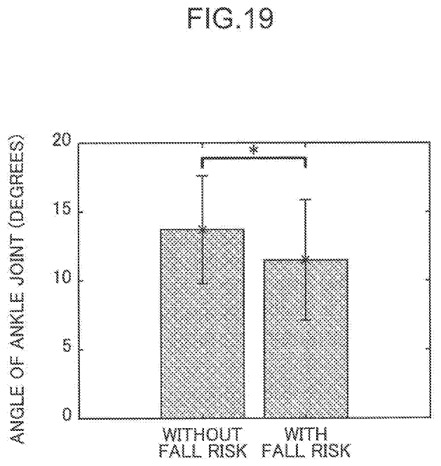

FIG. 19 is a view showing an average of mean values of time series data of the angle of the ankle joint of one foot of the subjects who do not have a fall risk in the period of 84% to 89% of one walking cycle and an average of mean values of time series data of the angle of the ankle joint of one foot of the subjects who have a fall risk in the period of 84% to 89% of one walking cycle in the sixth modification of the present embodiment.

As shown in FIG. 19, the average of mean values of time series data of the angle of the ankle joint of one foot of the subjects who do not have a fall risk in the period of 84% to 89% of one walking cycle was 13.7 degrees, and the average of mean values of time series data of the angle of the ankle joint of one foot of the subjects who have a fall risk in the period of 84% to 89% of one walking cycle was 11.4 degrees.

Thus, in the period of 84% to 89% of one walking cycle, the average of the mean values of time series data of the angle of the ankle joint of one foot of the subjects who have a fall risk is smaller than the average of the mean values of time series data of the angle of the ankle joint of one foot of the subjects who do not have a fall risk. Therefore, a value between an average of the mean values of time series data of the angle of the ankle joint of one foot in the period of 84% to 89% of one walking cycle of the subjects who have a fall risk and an average of the mean values of time series data of the angle of the ankle joint of one foot in the period of 84% to 89% of one walking cycle of the subjects who do not have a fall risk, having been experimentally obtained, may be stored in the memory 12 as the threshold value. The fall risk determination unit 113 may determine the presence or absence of a fall risk by comparing the mean value of time series data of the angle of one ankle joint of the subject in the period of 84% to 89% of one walking cycle with the threshold value stored in advance.

Subsequently, the walking parameter in the seventh modification of the present embodiment will be described.

The walking parameter in the seventh modification of the present embodiment may be a mean value of time series data of the vertical displacement of the waist in the first period of the stance phase of one leg and a mean value of time series data of the angle of the knee joint in the second period of the stance phase of one leg.

In the seventh modification of the present embodiment, similar to the above experiment, time series data of the vertical displacement of the waist of each of the plurality of subjects and time series data of the angle of one knee joint of each of the plurality of subjects were detected. In addition, in the experiment, one normalized walking cycle was divided into ten intervals, and the mean value of the vertical displacements of the waist and the mean value of the angles of one knee joint in one interval or two or more consecutive intervals were calculated for each subject. Then, a plurality of prediction models was created with whether or not the subject has a fall risk as an objective variable, and with the mean value of the vertical displacements of the waist and the mean value of the angles of one knee joint in one interval or two or more consecutive intervals as explanatory variables. The plurality of prediction models was evaluated by cross validation. Leave-one-out cross validation was adopted as the cross validation. Then, the ROC curve of each of the plurality of prediction models was calculated. Furthermore, the AUC value of the ROC curve of each of the plurality of prediction models was calculated, and the prediction model with the highest AUC value was selected.

In the seventh modification of the present embodiment, the prediction model with the mean value of the vertical displacements of the waist in the period of 1% to 40% of one walking cycle and the mean value of the angles of one knee joint in the period of 1% to 60% of one walking cycle as explanatory variables had the highest AUC value.

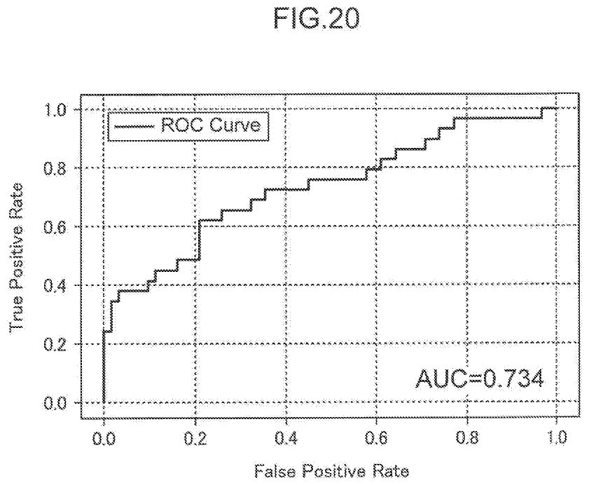

FIG. 20 is a view showing an ROC curve obtained as a result of determining the presence or absence of a fall risk using a prediction model in the seventh modification of the present embodiment.

The prediction model in the seventh modification of the present embodiment was created with whether or not the subject has a fall risk as an objective variable, and with the mean value of the vertical displacements of the waist in the period of 1% to 40% of one walking cycle and the mean value of the angles of one knee joint in the period of 1% to 60% of one walking cycle as explanatory variables. In FIG. 20, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 20 was obtained by plotting the true positive rate and the false positive rate of the prediction model created with the mean value of the vertical displacements of the waist in the period of 1% to 40% of one walking cycle and the mean value of the angles of one knee joint in the period of 1% to 60% of one walking cycle as explanatory variables. The AUC value of the ROC curve shown in FIG. 20 was 0.734. In this case, the mean value of the vertical displacements of the waist in the period of 1% to 40% of one walking cycle and the mean value of the angles of one knee joint in the period of 1% to 60% of one walking cycle are determined as walking parameters. In addition, the prediction model created with the mean value of the vertical displacements of the waist in the period of 1% to 40% of one walking cycle and the mean value of the angles of one knee joint in the period of 1% to 60% of one walking cycle as explanatory variables is determined as a prediction model to be used by the fall risk determination unit 113.

The walking parameter detection unit 112 detects time series data of the vertical displacement of the waist in the first period of the stance phase of one leg and time series data of the angle of the knee joint in the second period of the stance phase of one leg. The first period is a period of 1% to 40% of one walking cycle, and the second period is a period of 1% to 60% of one walking cycle. The walking parameter detection unit 112 detects time series data of the vertical displacement of the waist in the period of 1% to 40% of one walking cycle and time series data of the angle of one knee joint in the period of 1% to 60% of one walking cycle. In addition, the walking parameter detection unit 112 calculates the mean value of time series data of the vertical displacement of the waist in the period of 1% to 40% of one walking cycle and the mean value of time series data of the angle of one knee joint in the period of 1% to 60% of one walking cycle.

In particular, the memory 12 stores in advance a prediction model generated with the mean value of time series data of the vertical displacement of the waist in the first period of the stance phase of one leg and the mean value of time series data of the angle of the knee joint in the second period of the stance phase of one leg as input values and with whether or not the subject has a fall risk as an output value. In particular, the memory 12 stores in advance a prediction model generated with the mean value of the vertical displacements of the waist in the period of 1% to 40% of one walking cycle and the mean value of the angles of one knee joint in the period of 1% to 60% of one walking cycle as input values and with whether or not the subject has a fall risk as an output value.

The fall risk determination unit 113 determines the presence or absence of a fall risk of the subject by using the mean value of time series data of the vertical displacement of the waist and the mean value of time series data of the angle of the knee joint.

The fall risk determination unit 113 determines the presence or absence of a fall risk of the subject by using the mean value of time series data of the vertical displacement of the waist in the period of 1% to 40% of one walking cycle and the mean value of time series data of the angle of one knee joint in the period of 1% to 60% of one walking cycle. By inputting the mean value of time series data of the vertical displacement of the waist in the period of 1% to 40% of one walking cycle and the mean value of time series data of the angle of one knee joint in the period of 1% to 60% of one walking cycle into the prediction model, the fall risk determination unit 113 acquires, from the prediction model, a determination result indicating whether or not the subject has a fall risk.

Thus, the AUC value of the prediction model created using the vertical displacement of the waist in the stance phase in isolation was 0.733, and the AUC value of the prediction model created using the angle of the knee joint in the stance phase in isolation was 0.542. On the other hand, the AUC value of the prediction model created using the vertical displacement of the waist in the stance phase and the angle of the knee joint in the stance phase was 0.734. Accordingly, it is possible to determine the presence or absence of a fall risk more accurately in the prediction model created using the vertical displacement of the waist in the stance phase and the angle of the knee joint in the stance phase than in the prediction model created using each of the vertical displacement of the waist in the stance phase and the angle of the knee joint in the stance phase in isolation.

Subsequently, the walking parameters in the eighth modification of the present embodiment will be described.

The walking parameter in the eighth modification of the present embodiment may be a mean value of time series data of the vertical displacement of the waist in the period of 9% to 19% of one walking cycle of one leg and an angle of the knee joint at the time point of 35% of one walking cycle of one leg.

In the eighth modification of the present embodiment, similar to the above experiment, time series data of the vertical displacement of the waist of each of the plurality of subjects and time series data of the angle of one knee joint of each of the plurality of subjects were detected. In addition, a prediction model was created with whether or not the subject has a fall risk as an objective variable and with the mean value of the vertical displacements of the waist in the period of 9% to 19% of one walking cycle and the angle of one knee joint at the time point of 35% of one walking cycle as explanatory variables. The prediction model was evaluated by cross validation. Leave-one-out cross validation was adopted as the cross validation. Then, the ROC curve of the prediction model was calculated. Furthermore, the AUC value of the ROC curve of the prediction model was calculated.

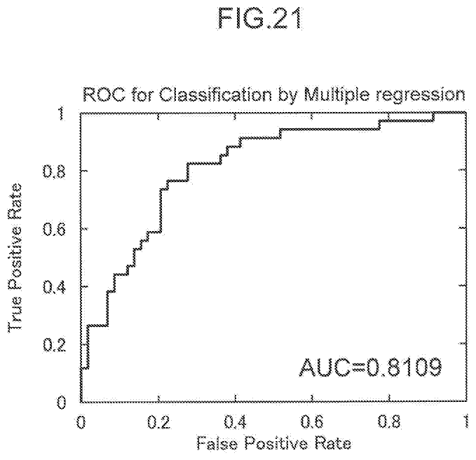

FIG. 21 is a view showing an ROC curve obtained as a result of determining the presence or absence of a fall risk using a prediction model in the eighth modification of the present embodiment.

The prediction model in the eighth modification of the present embodiment was created with whether or not the subject has a fall risk as an objective variable, and with the mean value of the vertical displacements of the waist in the period of 9% to 19% of one walking cycle and the angle of one knee joint at the time point of 35% of one walking cycle as explanatory variables. In FIG. 21, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 21 was obtained by plotting the true positive rate and the false positive rate of the prediction model created with the mean value of the vertical displacements of the waist in the period of 9% to 19% of one walking cycle and the angle of one knee joint at the time point of 35% of one walking cycle as explanatory variables. The AUC value of the ROC curve shown in FIG. 21 was 0.8109. In this case, the mean value of the vertical displacements of the waist in the period of 9% to 19% of one walking cycle and the angle of one knee joint at the time point of 35% of one walking cycle are determined as walking parameters. In addition, the prediction model created with the mean value of the vertical displacements of the waist in the period of 9% to 19% of one walking cycle and the angle of one knee joint at the time point of 35% of one walking cycle as explanatory variables is determined as a prediction model to be used by the fall risk determination unit 113.

The walking parameter detection unit 112 detects time series data of the vertical displacement of the waist in the first period of the stance phase of one leg and time series data of the angle of the knee joint in the second period of the stance phase of one leg. More specifically, the walking parameter detection unit 112 detects the time series data of the vertical displacement of the waist in the period of 9% to 19% of one walking cycle and the angle of one knee joint at the time point of 35% of one walking cycle. In addition, the walking parameter detection unit 112 calculates the mean value of time series data of the vertical displacement of the waist in the period of 9% to 19% of one walking cycle.

In particular, the memory 12 stores in advance a prediction model generated with the mean value of the vertical displacements of the waist in the period of 9% to 19% of one walking cycle and the angle of one knee joint at the time point of 35% of one walking cycle as input values and with whether or not the subject has a fall risk as an output value.

The fall risk determination unit 113 determines the presence or absence of a fall risk of the subject by using the mean value of time series data of the vertical displacement of the waist in the period of 9% to 19% of one walking cycle and the angle of one knee joint at the time point of 35% of one walking cycle. By inputting the mean value of time series data of the vertical displacement of the waist in the period of 9% to 19% of one walking cycle and the angle of one knee joint at the time point of 35% of one walking cycle into the prediction model, the fall risk determination unit 113 acquires, from the prediction model, a determination result indicating whether or not the subject has a fall risk.

Thus, the AUC value of the prediction model created using the vertical displacement of the waist in the period of 9% to 19% of one walking cycle in isolation was 0.8058, and the AUC value of the prediction model created using the angle of the knee joint at the time point of 35% of one walking cycle in isolation was 0.6242. On the other hand, the AUC value of the prediction model created using the vertical displacement of the waist in the period of 9% to 19% of one walking cycle and the angle of the knee joint at the time point of 35% of one walking cycle was 0.8109. Accordingly, it is possible to determine the presence or absence of a fall risk more accurately in the prediction model created using the vertical displacement of the waist in the period of 9% to 19% of one walking cycle and the angle of the knee joint at the time point of 35% of one walking cycle than in the prediction model created using each of the vertical displacement of the waist in the period of 9% to 19% of one walking cycle and the angle of the knee joint at the time point of 35% of one walking cycle in isolation.

Subsequently, the walking parameters in the ninth modification of the present embodiment will be described.

The walking parameter in the ninth modification of the present embodiment may be a mean value of time series data of the vertical displacement of the waist in the first period of the stance phase of one leg, a mean value of time series data of the vertical displacement of the waist in the second period of the swing phase of one leg, and a mean value of time series data of the angle of the ankle joint in the third period of the swing phase of one leg.

In the ninth modification of the present embodiment, similar to the above experiment, time series data of the vertical displacement of the waist of each of the plurality of subjects and time series data of the angle of one ankle joint of each of the plurality of subjects were detected. In addition, in the experiment, one normalized walking cycle was divided into ten intervals, and the mean value of the vertical displacements of the waist and the mean value of the angles of one ankle joint in one interval or two or more consecutive intervals were calculated for each subject. Then, a plurality of prediction models was created with whether or not the subject has a fall risk as an objective variable, and with the mean value of the vertical displacements of the waist and the mean value of the angles of one ankle joint in one interval or two or more consecutive intervals as explanatory variables. The plurality of prediction models was evaluated by cross validation. Leave-one-out cross validation was adopted as the cross validation. Then, the ROC curve of each of the plurality of prediction models was calculated. Furthermore, the AUC value of the ROC curve of each of the plurality of prediction models was calculated, and the prediction model with the highest AUC value was selected.

In the ninth modification of the present embodiment, the prediction model with the mean value of the vertical displacements of the waist in the period of 1% to 60% of one walking cycle, the mean value of the vertical displacements of the waist in the period of 61% to 80% of one walking cycle, and the mean value of the angles of one ankle joint in the period of 61% to 100% of one walking cycle as explanatory variables had the highest AUC value.

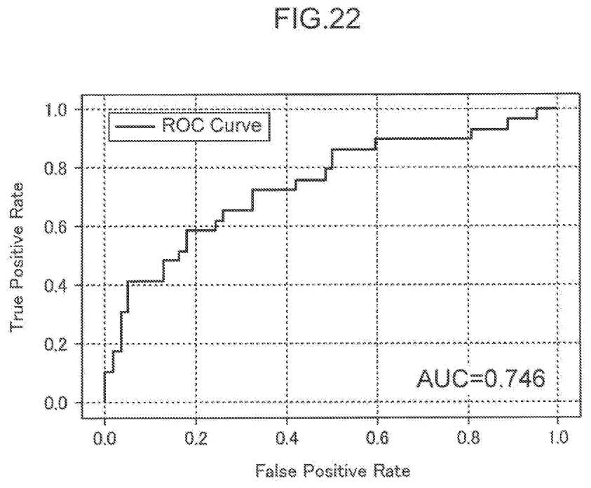

FIG. 22 is a view showing an ROC curve obtained as a result of determining the presence or absence of a fall risk using a prediction model in the ninth modification of the present embodiment.

The prediction model in the ninth modification of the present embodiment was created with whether or not the subject has a fall risk as an objective variable, and with the mean value of the vertical displacements of the waist in the period of 1% to 60% of one walking cycle, the mean value of the vertical displacements of the waist in the period of 61% to 80% of one walking cycle, and the mean value of the angles of one ankle joint in the period of 61% to 100% of one walking cycle as explanatory variables. In FIG. 22, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 22 was obtained by plotting the true positive rate and the false positive rate of the prediction model created with the mean value of the vertical displacements of the waist in the period of 1% to 60% of one walking cycle, the mean value of the vertical displacements of the waist in the period of 61% to 80% of one walking cycle, and the mean value of the angles of one ankle joint in the period of 61% to 100% of one walking cycle as explanatory variables. The AUC value of the ROC curve shown in FIG. 22 was 0.746. In this case, the mean value of the vertical displacements of the waist in the period of 1% to 60% of one walking cycle, the mean value of the vertical displacements of the waist in the period of 61% to 80% of one walking cycle, and the mean value of the angles of one ankle joint in the period of 61% to 100% of one walking cycle are determined as walking parameters. In addition, the prediction model created with the mean value of the vertical displacements of the waist in the period of 1% to 60% of one walking cycle, the mean value of the vertical displacements of the waist in the period of 61% to 80% of one walking cycle, and the mean value of the angles of one ankle joint in the period of 61% to 100% of one walking cycle as explanatory variables is determined as a prediction model to be used by the fall risk determination unit 113.

The walking parameter detection unit 112 detects time series data of the vertical displacement of the waist in the first period of the stance phase of one leg, time series data of the vertical displacement of the waist in the second period of the swing phase of one leg, and time series data of the angle of the ankle joint in the third period of the swing phase of one leg. The first period is a period of 1% to 60% of one walking cycle, the second period is a period of 61% to 80% of one walking cycle, and the third period is a period of 61% to 100% of one walking cycle. The walking parameter detection unit 112 detects time series data of the vertical displacement of the waist in the period of 1% to 60% of one walking cycle, time series data of the vertical displacement of the waist in the period of 61% to 80% of one walking cycle, and time series data of the angle of one ankle joint in the period of 61% to 100% of one walking cycle. In addition, the walking parameter detection unit 112 calculates the mean value of time series data of the vertical displacement of the waist in the period of 1% to 60% of one walking cycle, the mean value of time series data of the vertical displacement of the waist in the period of 61% to 80% of one walking cycle, and the mean value of time series data of the angle of one ankle joint in the period of 61% to 100% of one walking cycle.

In particular, the memory 12 stores in advance a prediction model generated with the mean value of time series data of the vertical displacement of the waist in the first period of the stance phase of one leg, the mean value of time series data of the vertical displacement of the waist in the second period of the swing phase of one leg, and the mean value of time series data of the angle of the ankle joint in the third period of the swing phase of one leg as input values and with whether or not the subject has a fall risk as an output value. In particular, the memory 12 stores in advance a prediction model generated with the mean value of the vertical displacements of the waist in the period of 1% to 60% of one walking cycle, the mean value of the vertical displacements of the waist in the period of 61% to 80% of one walking cycle, and the mean value of the angles of one ankle joint in the period of 61% to 100% of one walking cycle as input values and with whether or not the subject has a fall risk as an output value.

The fall risk determination unit 113 determines the presence or absence of a fall risk of the subject by using the mean value of time series data of the vertical displacement of the waist and the mean value of time series data of the angle of the ankle joint.

The fall risk determination unit 113 determines the presence or absence of a fall risk of the subject by using the mean value of time series data of the vertical displacement of the waist in the period of 1% to 60% of one walking cycle, the mean value of time series data of the vertical displacement of the waist in the period of 61% to 80% of one walking cycle, and the mean value of time series data of the angle of one ankle joint in the period of 61% to 100% of one walking cycle. By inputting the mean value of time series data of the vertical displacement of the waist in the period of 1% to 60% of one walking cycle, the mean value of time series data of the vertical displacement of the waist in the period of 61% to 80% of one walking cycle, and the mean value of time series data of the angle of one ankle joint in the period of 61% to 100% of one walking cycle into the prediction model, the fall risk determination unit 113 acquires, from the prediction model, a determination result indicating whether or not the subject has a fall risk.

Thus, the AUC value of the prediction model created using the vertical displacement of the waist in the stance phase in isolation was 0.733, and the AUC value of the prediction model created using the angle of the ankle joint in the swing phase in isolation was 0.595. On the other hand, the AUC value of the prediction model created using the vertical displacement of the waist in the first period of the stance phase, the vertical displacement of the waist in the second period of the swing phase, and the angle of the ankle joint in the third period of the swing phase was 0.746. Accordingly, it is possible to determine the presence or absence of a fall risk more accurately in the prediction model created using the vertical displacement of the waist in the first period of the stance phase, the vertical displacement of the waist in the second period of the swing phase, and the angle of the ankle joint in the third period of the swing phase than in the prediction model created using each of the vertical displacement of the waist in the stance phase and the angle of the ankle joint in the swing phase in isolation.

Subsequently, the walking parameters in the tenth modification of the present embodiment will be described.

The walking parameter in the tenth modification of the present embodiment may be a mean value of time series data of the angle of the knee joint in the first period of the stance phase of one leg and a mean value of time series data of the angle of the ankle joint in the second period of the swing phase of one leg.

In the tenth modification of the present embodiment, similar to the above experiment, time series data of the angle of one knee joint of each of the plurality of subjects and time series data of the angle of one ankle joint of each of the plurality of subjects were detected. In addition, in the experiment, one normalized walking cycle was divided into ten intervals, and the mean value of the angles of one knee joint and the mean value of the angles of one ankle joint in one interval or two or more consecutive intervals were calculated for each subject. Then, a plurality of prediction models was created with whether or not the subject has a fall risk as an objective variable, and with the mean value of the angles of one knee joint and the mean value of the angles of one ankle joint in one interval or two or more consecutive intervals as explanatory variables. The plurality of prediction models was evaluated by cross validation. Leave-one-out cross validation was adopted as the cross validation. Then, the ROC curve of each of the plurality of prediction models was calculated. Furthermore, the AUC value of the ROC curve of each of the plurality of prediction models was calculated, and the prediction model with the highest AUC value was selected.

In the tenth modification of the present embodiment, the prediction model with the mean value of the angles of one knee joint in the period of 1% to 60% of one walking cycle and the mean value of the angles of one ankle joint in the period of 61% to 100% of one walking cycle as explanatory variables had the highest AUC value.

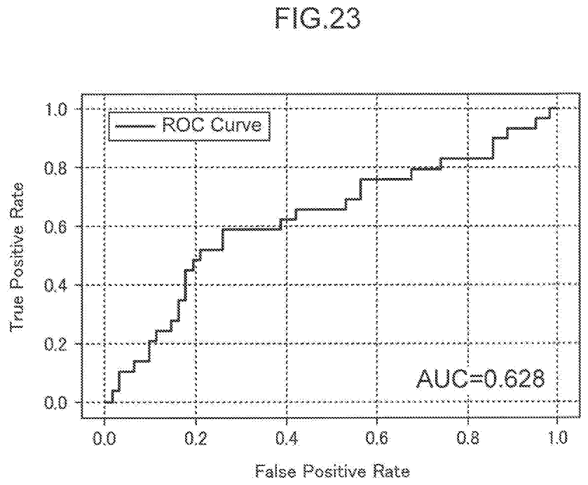

FIG. 23 is a view showing an ROC curve obtained as a result of determining the presence or absence of a fall risk using a prediction model in the tenth modification of the present embodiment.

The prediction model in the tenth modification of the present embodiment was created with whether or not the subject has a fall risk as an objective variable, and with the mean value of the angles of one knee joint in the period of 1% to 60% of one walking cycle and the mean value of the angles of one ankle joint in the period of 61% to 100% of one walking cycle as explanatory variables. In FIG. 23, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 23 was obtained by plotting the true positive rate and the false positive rate of the prediction model created with the mean value of the angles of one knee joint in the period of 1% to 60% of one walking cycle and the mean value of the angles of one ankle joint in the period of 61% to 100% of one walking cycle as explanatory variables. The AUC value of the ROC curve shown in FIG. 23 was 0.628. In this case, the mean value of the angles of one knee joint in the period of 1% to 60% of one walking cycle and the mean value of the angles of one ankle joint in the period of 61% to 100% of one walking cycle are determined as walking parameters. In addition, the prediction model created with the mean value of the angles of one knee joint in the period of 1% to 60% of one walking cycle and the mean value of the angles of one ankle joint in the period of 61% to 100% of one walking cycle as explanatory variables is determined as a prediction model to be used by the fall risk determination unit 113.

The walking parameter detection unit 112 detects time series data of the angle of the knee joint in the first period of the stance phase of one leg and time series data of the angle of the ankle joint in the second period of the swing phase of one leg. The first period is a period of 1% to 60% of one walking cycle, and the second period is a period of 61% to 100% of one walking cycle. The walking parameter detection unit 112 detects time series data of the angle of one knee joint in the period of 1% to 60% of one walking cycle and time series data of the angle of one ankle joint in the period of 61% to 100% of one walking cycle. In addition, the walking parameter detection unit 112 calculates the mean value of time series data of the angle of one knee joint in the period of 1% to 60% of one walking cycle and the mean value of time series data of the angle of one ankle joint in the period of 61% to 100% of one walking cycle.

In particular, the memory 12 stores in advance a prediction model generated with the mean value of time series data of the angle of the knee joint in the first period of the stance phase of one leg and the mean value of time series data of the angle of the ankle joint in the second period of the swing phase of one leg as input values and with whether or not the subject has a fall risk as an output value. In particular, the memory 12 stores in advance a prediction model generated with the mean value of the angles of one knee joint in the period of 1% to 60% of one walking cycle and the mean value of the angles of one ankle joint in the period of 61% to 100% of one walking cycle as input values and with whether or not the subject has a fall risk as an output value.

The fall risk determination unit 113 determines the presence or absence of a fall risk of the subject by using the mean value of time series data of the angle of the knee joint and the mean value of time series data of the angle of the ankle joint.

The fall risk determination unit 113 determines the presence or absence of a fall risk of the subject by using the mean value of time series data of the angle of one knee joint in the period of 1% to 60% of one walking cycle and the mean value of time series data of the angle of one ankle joint in the period of 61% to 100% of one walking cycle. By inputting the mean value of time series data of the angle of one knee joint in the period of 1% to 60% of one walking cycle and the mean value of time series data of the angle of one ankle joint in the period of 61% to 100% of one walking cycle into the prediction model, the fall risk determination unit 113 acquires, from the prediction model, a determination result indicating whether or not the subject has a fall risk.

Thus, the AUC values of the prediction models created using the angle of the knee joint in isolation and the angle of the ankle joint in isolation were 0.542 and 0.595, respectively, and the AUC value of the prediction model created using the angle of the knee joint and the angle of the ankle joint was 0.628. Accordingly, it is possible to determine the presence or absence of a fall risk more accurately in the prediction model created using the angle of the knee joint and the angle of the ankle joint than in the prediction model created using each of the angle of the knee joint and the angle of the ankle joint in isolation.

Subsequently, the walking parameters in the eleventh modification of the present embodiment will be described.

The walking parameter in the eleventh modification of the present embodiment may be a mean value of time series data of the vertical displacement of the waist in the first period of the stance phase of one leg, a mean value of time series data of the angle of the knee joint in the second period of the stance phase of one leg, and a mean value of time series data of the angle of the ankle joint in the third period of the swing phase of one leg.

In the eleventh modification of the present embodiment, similar to the above experiment, time series data of the vertical displacement of the waist of each of the plurality of subjects, time series data of the angle of one knee joint of each of the plurality of subjects, and time series data of the angle of one ankle joint of each of the plurality of subjects were detected. In addition, in the experiment, one normalized walking cycle was divided into ten intervals, and the mean value of the vertical displacements of the waist, the mean value of the angles of one knee joint, and the mean value of the angles of one ankle joint in one interval or two or more consecutive intervals were calculated for each subject. Then, a plurality of prediction models was created with whether or not the subject has a fall risk as an objective variable, and with the mean value of the vertical displacements of the waist, the mean value of the angles of one knee joint, and the mean value of the angles of one ankle joint in one interval or two or more consecutive intervals as explanatory variables. The plurality of prediction models was evaluated by cross validation. Leave-one-out cross validation was adopted as the cross validation. Then, the ROC curve of each of the plurality of prediction models was calculated. Furthermore, the AUC value of the ROC curve of each of the plurality of prediction models was calculated, and the prediction model with the highest AUC value was selected.

In the eleventh modification of the present embodiment, the prediction model with the mean value of the vertical displacements of the waist in the period of 1% to 60% of one walking cycle, the mean value of the angles of one knee joint in the period of 1% to 60% of one walking cycle, and the mean value of the angles of one ankle joint in the period of 61% to 100% of one walking cycle as explanatory variables had the highest AUC value.

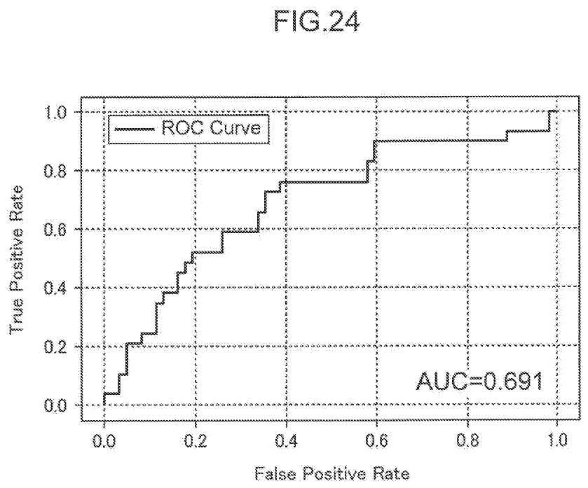

FIG. 24 is a view showing an ROC curve obtained as a result of determining the presence or absence of a fall risk using a prediction model in the eleventh modification of the present embodiment.

The prediction model in the eleventh modification of the present embodiment was created with whether or not the subject has a fall risk as an objective variable, and with the mean value of the vertical displacements of the waist in the period of 1% to 60% of one walking cycle, the mean value of the angles of one knee joint in the period of 1% to 60% of one walking cycle, and the mean value of the angles of one ankle joint in the period of 61% to 100% of one walking cycle as explanatory variables. In FIG. 24, the vertical axis represents the true positive rate, and the horizontal axis represents the false positive rate. The true positive rate indicates a ratio at which the prediction model has correctly determined a subject who has a fall risk as having a fall risk, and the false positive rate indicates a ratio at which the prediction model has incorrectly determined that a subject who does not have a fall risk as having a fall risk.

The ROC curve shown in FIG. 24 was obtained by plotting the true positive rate and the false positive rate of the prediction model created with the mean value of the vertical displacements of the waist in the period of 1% to 60% of one walking cycle, the mean value of the angles of one knee joint in the period of 1% to 60% of one walking cycle, and the mean value of the angles of one ankle joint in the period of 61% to 100% of one walking cycle as explanatory variables. The AUC value of the ROC curve shown in FIG. 24 was 0.691. In this case, the mean value of the vertical displacements of the waist in the period of 1% to 60% of one walking cycle, the mean value of the angles of one knee joint in the period of 1% to 60% of one walking cycle, and the mean value of the angles of one ankle joint in the period of 61% to 100% of one walking cycle are determined as walking parameters. In addition, the prediction model created with the mean value of the vertical displacements of the waist in the period of 1% to 60% of one walking cycle, the mean value of the angles of one knee joint in the period of 1% to 60% of one walking cycle, and the mean value of the angles of one ankle joint in the period of 61% to 100% of one walking cycle as explanatory variables is determined as a prediction model to be used by the fall risk determination unit 113.

The walking parameter detection unit 112 detects time series data of the vertical displacement of the waist in the first period of the stance phase of one leg, time series data of the angle of the knee joint in the second period of the stance phase of one leg, and time series data of the angle of the ankle joint in the third period of the swing phase of one leg. The first period is a period of 1% to 60% of one walking cycle, the second period is a period of 1% to 60% of one walking cycle, and the third period is a period of 61% to 100% of one walking cycle. The walking parameter detection unit 112 detects time series data of the vertical displacement of the waist in the period of 1% to 60% of one walking cycle, time series data of the angle of one knee joint in the period of 1% to 60% of one walking cycle, and time series data of the angle of one ankle joint in the period of 61% to 100% of one walking cycle.

In addition, the walking parameter detection unit 112 calculates the mean value of time series data of the vertical displacement of the waist in the period of 1% to 60% of one walking cycle, the mean value of time series data of the angle of one knee joint in the period of 1% to 60% of one walking cycle, and the mean value of time series data of the angle of one ankle joint in the period of 61% to 100% of one walking cycle.

In particular, the memory 12 stores in advance a prediction model generated with the mean value of time series data of the vertical displacement of the waist in the first period of the stance phase of one leg, the mean value of time series data of the angle of the knee joint in the second period of the stance phase of one leg, and the mean value of time series data of the angle of the ankle joint in the third period of the swing phase of one leg as input values and with whether or not the subject has a fall risk as an output value. In particular, the memory 12 stores in advance a prediction model generated with the mean value of the vertical displacements of the waist in the period of 1% to 60% of one walking cycle, the mean value of the angles of one knee joint in the period of 1% to 60% of one walking cycle, and the mean value of the angles of one ankle joint in the period of 61% to 100% of one walking cycle as input values and with whether or not the subject has a fall risk as an output value.

The fall risk determination unit 113 determines the presence or absence of a fall risk of the subject by using the mean value of time series data of the vertical displacement of the waist, the mean value of time series data of the angle of the knee joint, and the mean value of time series data of the angle of the ankle joint.

The fall risk determination unit 113 determines the presence or absence of a fall risk of the subject by using the mean value of time series data of the vertical displacement of the waist in the period of 1% to 60% of one walking cycle, the mean value of time series data of the angle of one knee joint in the period of 1% to 60% of one walking cycle, and the mean value of time series data of the angle of one ankle joint in the period of 61% to 100% of one walking cycle. By inputting the mean value of time series data of the vertical displacement of the waist in the period of 1% to 60% of one walking cycle, the mean value of time series data of the angle of one knee joint in the period of 1% to 60% of one walking cycle, and the mean value of time series data of the angle of one ankle joint in the period of 61% to 100% of one walking cycle into the prediction model, the fall risk determination unit 113 acquires, from the prediction model, a determination result indicating whether or not the subject has a fall risk.

Thus, the AUC values of the prediction models created using the angle of the knee joint in isolation and the angle of the ankle joint in isolation were 0.542 and 0.595, respectively, and the AUC value of the prediction model created using the vertical displacement of the waist, the angle of the ankle joint, and the angle of the knee joint was 0.691. Accordingly, it is possible to determine the presence or absence of a fall risk more accurately in the prediction model created using the vertical displacement of the waist, the angle of the knee joint, and the angle of the ankle joint than in the prediction model created using each of the angle of the knee joint and the angle of the ankle joint in isolation.

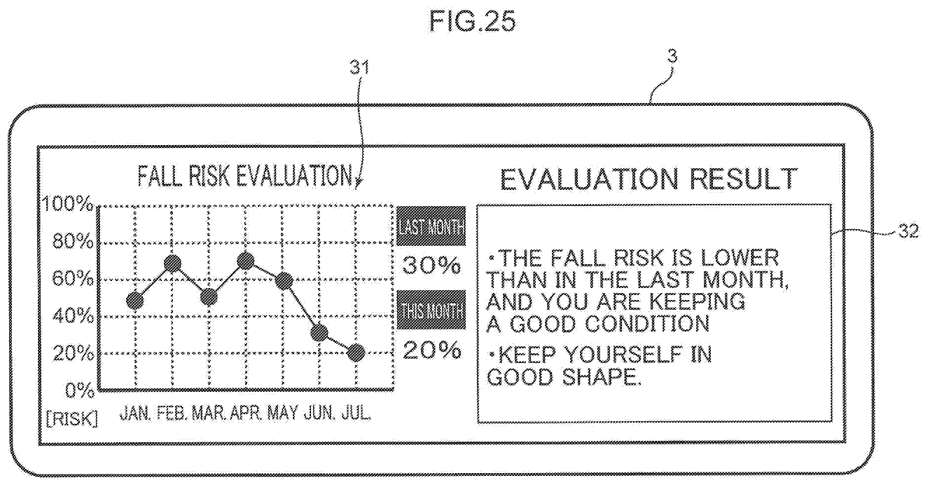

FIG. 25 is a view showing an example of an evaluation result screen displayed in the present embodiment.

The display unit 3 displays the evaluation result screen shown in FIG. 25. The evaluation result screen includes a fall risk evaluation presentation region 31 showing a past evaluation value of the fall risk and a current evaluation value of the fall risk, and an evaluation message 32. In the fall risk evaluation presentation region 31 of FIG. 25, evaluation of the fall risk is performed once a month, and the evaluation values of the fall risk for the past six months and the evaluation value of the fall risk for this month are displayed.

The evaluation value of the fall risk is a value indicating the fall risk level, calculated by the prediction model. The value indicating the fall risk level is represented by 0.0 to 1.0, for example. The evaluation result presentation unit 114 converts a value indicating the fall risk level into a percentage and presents it as an evaluation value of the fall risk.

It is to be noted that in a case where the past evaluation value of the fall risk is displayed together with the current evaluation value of the fall risk, the fall risk determination unit 113 stores the evaluation value of the fall risk in the memory 12.

In addition, the fall risk evaluation presentation region 31 may display, as an evaluation result, whether or not the subject has a fall risk.

In addition, the evaluation message 32 of “The fall risk is lower than in the last month, and you are keeping a good condition. Keep yourself in good shape.” is displayed. When the evaluation value of the fall risk of this month is lower than the evaluation value of the fall risk of the last month and the evaluation value of the fall risk of this month is lower than 0.5, the evaluation result presentation unit 114 reads the evaluation message 32 shown in FIG. 25 from the memory 12 and outputs it to the display unit 3.

It is to be noted that while in the present embodiment, the past evaluation values of the fall risk are displayed together with the current evaluation value of the fall risk, the present disclosure is not particularly limited to this, and only the current evaluation value of the fall risk may be displayed. In this case, the fall risk determination unit 113 is not required to store the evaluation value of the fall risk in the memory 12.

In addition, the camera 2 in the present embodiment may be a security camera provided in front of the entrance, a camera slave machine of a video intercom, or a monitoring camera provided in a room. In addition, the display unit 3 may be a display of a smartphone, a tablet computer, or a video intercom.

It is to be noted that while in the present embodiment, the walking parameter detection unit 112 extracts skeleton data based on the moving image data acquired from the camera 2, the present disclosure is not particularly limited thereto, and skeleton data may be extracted using a motion capture system. The motion capture system may be optical, magnetic, mechanical, or inertial sensor based. For example, in an optical motion capture system, a camera captures an image of a subject with a marker attached to a joint and detects the position of the marker from the captured image. The walking parameter detection unit 112 acquires the skeleton data of the subject from the position data detected by the motion capture system. As the optical motion capture system, for example, a three-dimensional motion analysis device manufactured by Inter Reha Co., Ltd. is available.

In addition, the motion capture system may include a depth sensor and a color camera, and the motion capture system may automatically extract position information of a joint point of the subject from an image and detect the attitude of the subject. In this case, the subject does not need to attach the marker. As such a motion capture system, for example, Kinect manufactured by Microsoft Corporation is available.

In measurement of walking motion using a motion capture system, it is preferable that the angle of the ankle joint, the angle of the kneejoint, or the vertical displacement of the waist in the walking motion be extracted from the position coordinates, and the feature amount of the walking motion be detected from the extracted angle or displacement.

It is to be noted that, in each of the above embodiments, each component may be configured by dedicated hardware or may be realized by executing a software program suitable for each component. Each component may be realized by a program execution unit such as a CPU or a processor reading and executing a software program recorded in a recording medium such as a hard disk or a semiconductor memory.

Some or all of the functions of the device according to the embodiment of the present disclosure are realized as a large scale integration (LSI), which is typically an integrated circuit. These may be individually integrated into one chip, or may be integrated into one chip so as to include some or all of them. In addition, the integrated circuit is not limited to LSI, and may be realized by a dedicated circuit or a general-purpose processor. A field programmable gate array (FPGA), which can be programmed after manufacturing the LSI, or a reconfigurable processor, which can reconfigure the connection and setting of the circuit cell inside the LSI, may be used.

In addition, some or all of the functions of the device according to the embodiment of the present disclosure may be realized by a processor such as a CPU executing a program.

In addition, all of the numerals used above are merely examples for specifically describing the present disclosure, and the present disclosure is not limited to the exemplified numerals.

In addition, the order of executing the steps shown in the flowchart is an example for the purpose of specifically describing the present disclosure, and may be any order other than the above as long as a similar effect is obtained. In addition, some of the above steps may be executed simultaneously (parallel) with other steps.

Since the technology according to the present disclosure can simply and highly accurately evaluate the fall risk, it is useful for the technology of evaluating the fall risk based on the walking motion of a subject.

This application is based on U.S. Provisional application No. 62/893,304 filed in United States Patent and Trademark Office on Aug. 29, 2019 and Japanese Patent application No. 2020-023433 filed in Japan Patent Office on Feb. 14, 2020, the contents of which are hereby incorporated by reference.

Although the present invention has been fully described by way of example with reference to the accompanying drawings, it is to be understood that various changes and modifications will be apparent to those skilled in the art. Therefore, unless otherwise such changes and modifications depart from the scope of the present invention hereinafter defined, they should be construed as being included therein.

1. A fall risk evaluation method in a fall risk evaluation device that evaluates a fall risk based on a walking motion of a subject, the fall risk evaluation method comprising:
acquiring walking data related to walking of the subject;
detecting, from the walking data, at least one of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase; and
determining a fall risk of the subject by using at least one of the vertical displacement of the waist in the stance phase, the vertical displacement of the waist in the swing phase, the angle of the knee joint in the stance phase, and the angle of the ankle joint in the swing phase.

2. The fall risk evaluation method according to claim 1, wherein
in the detection, time series data of the vertical displacement of the waist in a predetermined period of the stance phase is detected, and
in the determination, the fall risk of the subject is determined by using a mean value of the time series data of the vertical displacement of the waist.

3. The fall risk evaluation method according to claim 2, wherein
on a condition that a period from when one foot of the subject touches a ground to when the one foot touches the ground again is expressed as one walking cycle and the one walking cycle is expressed by 1% to 100%, the predetermined period is a period of 1% to 60% of the one walking cycle.

4. The fall risk evaluation method according to claim 2, wherein
on a condition that a period from when one foot of the subject touches a ground to when the one foot touches the ground again is expressed as one walking cycle and the one walking cycle is expressed by 1% to 100%, the predetermined period is a period of 9% to 19% of the one walking cycle.

5. The fall risk evaluation method according to claim 1, wherein
in the detection, time series data of the vertical displacement of the waist in a predetermined period of the swing phase is detected, and
in the determination, the fall risk of the subject is determined by using a mean value of the time series data of the vertical displacement of the waist.

6. The fall risk evaluation method according to claim 5, wherein
on a condition that a period from when one foot of the subject touches a ground to when the one foot touches the ground again is expressed as one walking cycle and the one walking cycle is expressed by 1% to 100%, the predetermined period is a period of 61% to 100% of the one walking cycle.

7. The fall risk evaluation method according to claim 1, wherein
in the detection, time series data of an angle of the knee joint in a predetermined period of the stance phase is detected, and
in the determination, the fall risk of the subject is determined by using a mean value of the time series data of the angle of the knee joint.

8. The fall risk evaluation method according to claim 7, wherein
on a condition that a period from when one foot of the subject touches a ground to when the one foot touches the ground again is expressed as one walking cycle and the one walking cycle is expressed by 1% to 100%, the predetermined period is a period of 1% to 60% of the one walking cycle.

9. The fall risk evaluation method according to claim 7, wherein
on a condition that a period from when one foot of the subject touches a ground to when the one foot touches the ground again is expressed as one walking cycle and the one walking cycle is expressed by 1% to 100%, in the determination, the fall risk of the subject is determined by using the angle of the knee joint at a time point of 35% of the one walking cycle.

10. The fall risk evaluation method according to claim 1, wherein
in the detection, time series data of the angle of the ankle joint in a predetermined period of the swing phase is detected, and
in the determination, the fall risk of the subject is determined by using a mean value of the time series data of the angle of the ankle joint.

11. The fall risk evaluation method according to claim 10, wherein
on a condition that a period from when one foot of the subject touches a ground to when the one foot touches the ground again is expressed as one walking cycle and the one walking cycle is expressed by 1% to 100%, the predetermined period is a period of 61% to 100% of the one walking cycle.

12. The fall risk evaluation method according to claim 10, wherein
on a condition that a period from when one foot of the subject touches a ground to when the one foot touches the ground again is expressed as one walking cycle and the one walking cycle is expressed by 1% to 100%, the predetermined period is a period of 84% to 89% of the one walking cycle.

13. The fall risk evaluation method according to claim 1, wherein
in the detection, time series data of the vertical displacement of the waist in a first period of the stance phase and time series data of the angle of the knee joint in a second period of the stance phase are detected, and
in the determination, the fall risk of the subject is determined by using a mean value of the time series data of the vertical displacement of the waist in the first period and a mean value of the time series data of the angle of the knee joint in the second period.

14. The fall risk evaluation method according to claim 1, wherein
in the detection, time series data of the vertical displacement of the waist in a first period of the stance phase, time series data of the vertical displacement of the waist in a second period of the swing phase, and time series data of the angle of the ankle joint in a third period of the swing phase are detected, and
in the determination, the fall risk of the subject is determined by using a mean value of the time series data of the vertical displacement of the waist in the first period, a mean value of the time series data of the vertical displacement of the waist in the second period, and a mean value of the time series data of the angle of the ankle joint in the third period.

15. The fall risk evaluation method according to claim 1, wherein
in the detection, time series data of the angle of the knee joint in a first period of the stance phase and time series data of the angle of the ankle joint in a second period of the swing phase are detected, and
in the determination, the fall risk of the subject is determined by using a mean value of the time series data of the angle of the knee joint in the first period and a mean value of the time series data of the angle of the ankle joint in the second period.

16. The fall risk evaluation method according to claim 1, wherein
in the detection, time series data of the vertical displacement of the waist in a first period of the stance phase, time series data of the angle of the knee joint in a second period of the stance phase, and time series data of the angle of the ankle joint in a third period of the swing phase are detected, and
in the determination, the fall risk of the subject is determined by using a mean value of the time series data of the vertical displacement of the waist in the first period, a mean value of the time series data of the angle of the knee joint in the second period, and a mean value of the time series data of the angle of the ankle joint in the third period.

17. The fall risk evaluation method according to claim 1, wherein
in the determination, it is determined that the subject has the fall risk when the vertical displacement of the waist in the stance phase is smaller than a threshold value, when the vertical displacement of the waist in the swing phase is smaller than a threshold value, when the angle of the knee joint in the stance phase is smaller than a threshold value, or when the angle of the ankle joint in the swing phase is smaller than a threshold value.

18. The fall risk evaluation method according to claim 1, wherein
in the determination, whether or not the subject has the fall risk is determined by inputting at least one of the vertical displacement of the waist in the stance phase, the vertical displacement of the waist in the swing phase, the angle of the knee joint in the stance phase, and the angle of the ankle joint in the swing phase that have been detected into a prediction model generated with at least one of the vertical displacement of the waist in the stance phase, the vertical displacement of the waist in the swing phase, the angle of the knee joint in the stance phase, and the angle of the ankle joint in the swing phase as an input value, and with whether or not the subject has the fall risk as an output value.

19. A fall risk evaluation device that evaluates a fall risk based on a walking motion of a subject, the fall risk evaluation device comprising:
an acquisition unit that acquires walking data related to walking of the subject;
a detection unit that detects, from the walking data, at least one of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase; and
a determination unit that determines a fall risk of the subject by using at least one of the vertical displacement of the waist in the stance phase, the vertical displacement of the waist in the swing phase, the angle of the knee joint in the stance phase, and the angle of the ankle joint in the swing phase.

20. A non-transitory computer-readable recording medium in which a fall risk evaluation program that evaluates a fall risk based on a walking motion of a subject is recorded, wherein
the non-transitory computer-readable recording medium causes a computer to function so as to acquire walking data related to walking of the subject,
so as to detect, from the walking data, at least one of a vertical displacement of a waist of the subject in a stance phase of one leg of the subject, a vertical displacement of the waist of the subject in a swing phase of the one leg, an angle of a knee joint of the one leg in the stance phase, and an angle of an ankle joint of one foot in the swing phase, and
so as to determine a fall risk of the subject by using at least one of the vertical displacement of the waist in the stance phase, the vertical displacement of the waist in the swing phase, the angle of the knee joint in the stance phase, and the angle of the ankle joint in the swing phase.
